(19) United States
(12) Patent Application Publication
(10) Pub. No.: US 2013/0066237 A1
(43) Pub. Date: Mar. 14, 2013

(54) METHODS AND DEVICES FOR INFLAMMATION TREATMENT

(71) Applicants: Michael H. Smotrich, Andover, MA (US); Ilya Yaroslavsky, North Andover, MA (US); Gregory B. Altshuler, Lincoln, MA (US); Richard Cohen, Sherborn, MA (US)

(72) Inventors: Michael H. Smotrich, Andover, MA (US); Ilya Yaroslavsky, North Andover, MA (US); Gregory B. Altshuler, Lincoln, MA (US); Richard Cohen, Sherborn, MA (US)

(73) Assignee: PALOMAR MEDICAL TECHNOLOGIES, INC., Burlington, MA (US)

(21) Appl. No.: 13/540,455

(22) Filed: Jul. 2, 2012

Related U.S. Application Data
(60) Provisional application No. 61/532,934, filed on Sep. 9, 2011.

(51) Int. Cl. A61N 5/06; A61N 5/00; A61N 7/00

(52) U.S. Cl. 601/2; 604/20; 607/1; 607/88; 607/96

(57) ABSTRACT

Methods and devices are disclosed for controlled mediation and/or improvement of inflammation, inflammation associated with pain, and pain by delivering non-ablative thermal tissue damage to portions of a region of tissue including a volume of inflamed tissue, thereby activating the immune systems pain relief response to the tissue damage.

### RELATED APPLICATIONS

This application claims the benefit of U.S. Provisional Application No. 61/532,934, filed Sep. 9, 2011, which is incorporated herein by reference in its entirety.

### FIELD OF THE INVENTION

The devices and methods disclosed herein relate to inflammation treatment and/or pain treatment by irradiation of tissue (e.g., inflamed tissue) with radiation sources, including, but not limited to, ultrasound radiation, radio frequency radiation, and/or optical radiation having wavelengths in the ultraviolet, visible, and infrared ranges.

### BACKGROUND OF THE INVENTION

The sensation of pain is a complex phenomenon as well as a debilitating condition when it is persistent. Pain can be associated with tissue inflammation. One commonly accepted theory is that during the inflammatory response to injury or illness, afferent neurons are the initiators and providers of the pathway whereby nociceptive impulses are transmitted to the brain where they are perceived as pain.

### Exogenous Opioid Treatments

Acute or persistent pain can be alleviated by administration (e.g., orally and/or intravenously) of exogenous opioids. Exogenous opioids are fast acting in providing pain relief so they are excellent for surgery where need is immediate. However, the use of exogenous opioids is often limited by side effects, such as nausea, clouding of consciousness, depression of breathing, constipation, tolerance, and addiction. Thus, there are many downsides to use of exogenous opioids for chronic pain issues. Negative side effects of exogenous opioids are believed to be due, at least in part, to penetration of the opioids into the central nervous system. Currently the goal of several pharmaceutical companies is to find an exogenous opioid with a molecular configuration that provides the needed relief and/or analgesia, but remains in the peripheral nervous system and cannot enter the central nervous system thereby obviating/avoiding the negative side effects associated therewith.

### Endogenous Opioid Treatments

It has been known for many years that natural opioids, endogenous opioids, are produced by the body during the inflammatory phase of the immune system's “wound healing” response to injury. These endogenous opioids, e.g., endorphins, provide a level of natural pain mediation and/or relief and suggest that the immune system can play central role in orchestrating pain control. Specifically, it is believed that leukocytes migrating to areas of inflammation release endogenous opioids, which are taken up by the afferent neurons to block pain signals.

Various alternatives to pharmaceutical pain suppression have been proposed including acupuncture, electrical stimulation of nerves (e.g., Transcutaneous, Electrical Neuron Stimulation or TENS) and ultrasound.

The mechanisms that are involved in acupuncture are a subset of the mechanisms to stimulate the endogenous opiates. Acupuncture does the job of an analgesic, specifically, it provides stress induced analgesia. Some of the downsides of acupuncture include (1) probability of infection (2) probability of harm in treatment and (3) reliance on the skill level of the practitioner, because acupuncturists need to be highly skilled.

TENS provides electrical stimulation on the surface of the flesh. Like acupuncture a TENS practitioner needs to be highly skilled, at least in part, because the procedure can cause a high pain level.

Electrically assisted acupuncture can be described as a combination of acupuncture and TENS where the acupuncture needles are charged. Electrically assisted acupuncture requires some technique mastery to have success. Acupuncture with electrical assistance is conducted at from about 2 Hz to about 100 Hz, different effects can be achieved with different pulse widths, but penetration depth is not controlled, rather, it is subject to practitioner skill and/or judgment. Electrically assisted acupuncture requires a highly skilled practitioner at least because it can cause a high pain level.

### SUMMARY OF THE INVENTION

The prior treatment methods that promote endogenous opioids (e.g., acupuncture, TENS, electrically assisted acupuncture) are limited in that they are painful and/or depend on a high level of practitioner skill.

The use of light has also been proposed for pain relief. See, for example, Wong, U.S. Pat. No. 5,640,978, entitled “Method For Pain Relief Using Low Power Laser Light,” Friedman, U.S. Pat. No. 6,450,170, entitled “Treatment Of Migraine, Post-Traumatic Headache, Tension-Type Headaches, Atypical Facial Pain, Cervical Pain And Muscle Spasm,” and Masotti et al., U.S. Pat. No. 6,527,797, entitled “Laser Device For Treatment Of Painful Symptomatologies And Associated Method.”

None of these therapies previously proposed for pain suppression by use of EMR or by promoting endogenous opioids involve irradiation to damage tissue and/or so-called “fractional” irradiation to damage regions of tissue.

In the cosmetic field, methods and devices for the treatment of various skin conditions have been developed that irradiate or cause damage in a portion of the tissue area and/or volume being treated. These methods and devices have become known as fractional technology. Fractional technology is thought to be a relatively safe method of treatment of skin for cosmetic purposes, because tissue damage occurs within smaller sub-volumes or islets within the larger volume of tissue being treated. The tissue surrounding the islets is spared from the damage. See, for example, U.S. Pat. No. 6,997,923 by Anderson et al., entitled “Method and Apparatus for EMR Treatment,” U.S. Patent Application Pub. No. 20080214988 by Altshuler et al., entitled “Methods And Devices For Fractional Ablation Of Tissue,” and U.S. Patent Application Pub. No. 20100145321, by Altshuler et al., entitled “Methods And Products For Producing Lattices Of EMR-Treated Islets In Tissues, And Uses Therefor,” the disclosures of which are incorporated herein in their entireties. Examples of devices that have been used by professionals to treat the skin using fractional irradiation include the Palomar® 1540 Fractional Handpiece, the Palomar® PaloVia® Skin Renewing Laser, the Reliant Fraxel® SR Laser and similar devices by ActiveFX, Alma Lasers, Iridex, and Reliant Technologies. The Palomar® PaloVia® Skin Renewing Laser uses fractional irradiation to treat the skin in a home use setting with the consumer doing a self-treatment.

The present disclosure describes fractional irradiation methods and devices to treat and/or control pain, pain associated with inflammation, and inflammation. In various embodiments, examples of which are described in greater detail below, improved devices and systems are provided for treating pain by producing lattices of EMR-treated islets in target tissue regions (e.g., volumes and/or regions of inflamed tissue and/or adjacent to (including above) volumes of inflamed tissue). The methods of the present disclosure include the application of radiation, which is applied in a multitude of points (e.g., fractionally) to a patient's tissue in order to mediate inflammation, improve the perception of pain, and/or improve pain associated with inflammation.

Millions of years of human evolution have provided us with an intrinsic opioid based system capable of dealing with a broad spectrum of pain conditions. In accordance with the present disclosure, it is proposed that fractionally delivered energy is capable of providing analgesia to pain emanating from inflamed tissue. Candidate fractional systems must be capable of producing micro damage in inflamed tissue and/or tissue adjacent inflamed tissue and be based on technology scalable to both clinical applications and at home applications. Suitable fractional technologies can include, for example: optical, radio frequency, acoustic, and/or microwave radiation or any radiation that is capable of wounding tissue in a manner leading to pain control.

Furthermore, it is the applicants' belief that fractional treatment of inflammation, pain, and pain associated with inflammation is a non-invasive treatment alternative to medication-based treatment with the very important benefit of minimal to no side effects. The science community has studied the role of endogenous opioids in controlling pain. There is a homeostatic level of endogenous opioids in the human body that are produced to handle occasional injury or disruption and hence provide a type of natural pain control. Such endogenous opioids work best to control pain when the source of the pain is inflamed tissue. There are limits to this natural pain control capability. For example, when the body has an acute injury and/or develops a chronic condition for example arthritis, it usually not possible for this natural pain control system to cope with the pain situation. It is beyond the capability of the homeostatic pain control capacity, requiring more than the homeostatic level of endogenous opioids to achieve relief. The present disclosure, presents a method for increasing the capability of the endogenous opiate based pain control system with a goal of providing a level of endogenous opiates consistent with an acceptable quality of life.

Once columns of damage (e.g., fractional columns of damage) are established in the area adjacent the inflamed tissue (e.g., above the inflamed tissue) and/or in the inflamed tissue, the immune system (IS) controlled wound healing process immediately starts. This complex and well-coordinated wound healing process is orchestrated by the release of cytokines, signaling proteins, which trigger a sequence of events resulting in complete healing of the fractional damage. Included in the above cytokine mix, two cytokines, tumor necrosis factor alpha (TNF-alpha) and Interleukin (IL-1) are responsible for the production of corticotropin releasing hormone (CRH), which controls the amount of endogenous opiate available to control extraordinary pain conditions.

A further goal of the disclosed methods and devices for treatment of pain and inflammation are to avoid having an enhanced supply of endogenous opioids delivered to the central nervous system, following an acute injury and by so doing, contributing to unwanted side effects generally associated with exogenous opioid use. Control of allocation of endogenous opioids primarily to the peripheral nervous system is controlled at least in part by the temporal control of the treatment sequence. Since central nervous system opioid receptors degrade after several hours while peripheral nervous system receptors remain vital for an extended time period (e.g., peripheral nervous system receptors can remain vital for many days), by delaying the start of treatment for a period of time (e.g., approximately six hours) from the beginning of the presence of the pain and/or inflammation and/or inflammation associated with pain the opioids can be delivered with minimal to no side effects to the source of pain and/or inflammation and/or inflammation associated with pain.

The pain response cascade is a complex electrochemical/physiological process controlled by the Immune System. Specifically, the process of pain control is implemented by the peripheral nervous system. Immune System activity is initiated once damage columns, produced by fractional treatment, are recognized as a foreign body by the immune system. As a result, the immune systems pain relief response (including CRH release) is activated. The key is to create the damage (e.g., the foreign body) in the correct region of the body that is inflamed and/or adjacent to the inflamed tissue (e.g., above the inflamed tissue) and is, in some embodiments is a chronic source of pain (e.g., is chronically inflamed). During the inflammatory phase there is a production of endogenous opiates that can be used to mediate (e.g., slow and/or stop) the transmission of an electrochemical (action potential) signal resulting in mediation of pain in the peripheral nervous system. Thus, introduction of the foreign body via fractional treatment causing fractional damage is part of the pain relief procedure. The signals caused by introducing fractional columns combine to tell the body how to respond.

Fractional device(s) can exert control over the pain control function of the immune system and the nervous system by intentionally stressing the region that includes the inflamed tissue that produces pain. This can include intentionally stressing the inflamed tissue that produces pain and tissue that is adjacent (e.g., above) the inflamed tissue that produces pain. By producing repetitive thermal stresses, a fractional device, by way of the Gate Theory of Melzack and Wall, can mediate the sensation of pain. By providing stress in the area adjacent and/or including the inflamed tissue, a fractional device can, in conjunction with the immune system, initiate the delivery of opioid peptides to opioid receptors on the afferent neurons thereby mediating pain signal transmission. Leukocytes are immune system cells that constantly circulate in the blood stream and provide the delivery of endogenous opioids in the form of opioid-peptides. The immune system signals the location of the inflamed tissue initiating the process (i.e., extravasation) resulting in the delivery of the opioid bearing leukocyte to the pain source.

These methods may be employed in professional settings, by a licensed practitioner, or in a home use setting by the person suffering from pain and/or inflammation. In one embodiment, a home use non-ablative fractional device (e.g., a device such as a PaloVia® Skin Renewing Laser, a non-ablative fractional device sold for cosmetic treatments) can be used to cause coagulated damage columns in the tissue. The subject's immune system recognizes these damage columns as a foreign body or multiple foreign bodies and as a result the immune systems pain relief response is activated. The key to pain and/or inflammation treatment is to create the columns of damage that provide a foreign body or foreign bodies in the region of the body that includes the inflamed tissue. The region of the body may be, for example, a chronic source of pain such as, for example, joint pain. The introduction of the foreign body is part of the pain relief procedure.

In accordance with the prevailing theory of pain treatment, CRH allows the release of the opioid peptide from leukocyte. Stressing the tissue via introduction of fractional columns releases the peptide enabling the analgesic effect of the leukocyte by controlling CRH release by activating the immune systems pain relief response. This is a completely endogenous process controlled by forming fractional volumes of damage in the region that includes the inflamed tissue (e.g., in portions of tissue adjacent the inflamed volume and/or in the inflamed volume itself and/or in portions of the tissue adjacent the inflamed volume and in the inflamed volume). Validity (or lack thereof) of this theory does not, however, affect in any way the scope of the present disclosure.

The analgesic effect of the released peptide (e.g., the endogenous opioids) can also be controlled and/or enhanced by controlling and/or adjusting the level of CRH available in the body of the subject. In one embodiment, additional CRH is introduced to the body of the subject by, for example, injecting exogenous CRH into the inflamed volume and/or in the region of tissue that includes the inflamed tissue. It is anticipated that delivery of exogenous CRH could be accomplished by employing fractional systems not limited to the fractional pain treatment system, for example, to enable delivery of the CRH via the treated tissue (see, e.g., United States Publication No. 2006/0004347 A1 entitled “Methods and Products for Producing Lattices of EMR-Treated Islets in Tissues, and Uses Therefore” and United States Publication No. US-2009-0069741-A1 entitled “Methods and Devices for Fractional Ablation of Tissue For Substance Delivery”). The level of CRH may also be enhanced by use of available oral medications containing CRH.

In one aspect, the disclosure relates to a method for treating inflammation and/or pain the method includes determining a location of a volume of inflamed tissue and applying radiation suitable for damaging tissue (e.g., stressing tissue) to portions of the volume where a damaged portion of the volume is separated from another damaged portion by a non-treated portion of the volume. The volume of inflamed tissue can be determined by the subject feeling that the location is tender to the touch. Methods for treating inflammation and/or pain can also include applying radiation suitable for damaging tissue to the region that includes the inflamed tissue (e.g., in portions of tissue adjacent the inflamed tissue volume and/or in the inflamed tissue volume itself and/or in portions of the tissue adjacent the inflamed tissue volume and in the inflamed tissue volume).

Suitable radiation can include, for example, electromagnetic radiation. The radiation can be, for example, at least one of optical radiation, ultrasound radiation, and radio frequency radiation.

In accordance with this method, a multitude of micro columns of damage are created in the body, the columns typically (but not necessarily) extending from the skin surface to a certain depth. The depth, diameter, and density of the columns are precisely controlled by the treatment device. Dependence of the depth and diameter on the energy of the pulse is illustrated by FIGS. 16A-D and 17. Preferred treatment parameters are exemplified by Table 1. In one embodiment, the method also includes introducing a corticotropin-releasing hormone to the body of the subject. The corticotropin-releasing hormone can be a topical that is disposed on the location of the volume prior to, after, or simultaneous with applying radiation to the volume. In some embodiments, corticotropin-releasing hormone is injected into the location of the volume. In other embodiments, the corticotropin-releasing hormone is ingested prior to applying radiation. In one embodiment, radiation suitable for ablating tissue (e.g., with wavelengths from about 250 nm to about 12,000 nm) is applied to portions such that an ablated portion of the volume is separated from another ablated portion by a non-treated portion of the volume. A corticotropin-releasing hormone is disposed on the location of the volume (e.g., prior to, simultaneous with, or after forming the ablated portions of the volume). In some embodiments, the method is employed to treat a volume of inflamed tissue in a subject having a substantially healthy immune system. In some embodiments, the volume of tissue has been inflamed for at least 360 minutes.

In another aspect, the disclosure relates to a method for treating inflammation or pain that includes applying radiation suitable for damaging tissue to portions of a volume of tissue, where a damaged portion of the volume is separated from another damaged portion by a non-treated portion of the volume. The method also includes introducing a corticotropin-releasing hormone to the body of the subject. The corticotropin-releasing hormone may be in a topical (e.g., Acthar Gel) and it may be disposed on the location of the volume prior to, simultaneous with, or after applying radiation to the volume of tissue. In some embodiments, the corticotropin-releasing hormone is introduced to the body of the subject by, for example, injection into the location of the volume. In other embodiments, the corticotropin-releasing hormone is ingested by the subject prior to applying radiation. The method is expected to have improved efficacy when it is employed to treat inflammation or pain in a subject having substantially healthy immune system. In some embodiments, the volume of tissue has been inflamed for at least 360 minutes.

Sources of radiation may be, for example, electromagnetic radiation. Suitable sources of radiation can be at least one of optical radiation, ultrasound radiation, and radio frequency radiation. In some embodiments, the volume of inflamed tissue is determined by the subject by the subject feeling that the location of the volume of inflamed tissue is tender to the touch.

In another aspect, the disclosure relates to a method for treating chronic pain by determining a location of a volume of inflamed tissue that is associated with the chronic pain and delivering non-ablative thermal tissue damage to a depth of at least 50 microns to at least a portion of the inflamed tissue volume itself, to at least a portion of tissue adjacent the inflamed tissue volume and/or to at least a portion of the tissue adjacent the inflamed tissue volume and to at least a portion of the inflamed tissue volume. Optionally, the non-ablative thermal tissue damage is delivered to a depth of at least 100 microns. In some embodiments, the thermal tissue damage is to a depth of from about 100 microns to about 500 microns, from about 50 microns to about 250 microns, from about 50 microns to about 1000 microns. The method can include the step of determining the need for additional chronic pain treatment upon the resolution of the thermal tissue damage previously delivered. Optionally, a thermal tissue damage portion may be separated from another thermal tissue damage portion by a non-treated portion of the volume.

In another aspect, the disclosure relates to a device for treating inflammation or pain including an inflammation detector for detecting inflammation in a region of tissue and a source of radiation configured to generate radiation to damage a volume of tissue. The device also includes an optical path that delivers radiation from the source to the volume of tissue to form a damaged portion of the volume separated from another damaged portion of the volume by a non-treated portion of the volume. The inflammation detector can include at least one of a thermometer, a medical IR thermal camera, a thermally sensitive film, a video camera, and an ultrasound inflammation detector. In one embodiment, the optical path delivers radiation solely to the region of tissue containing inflammation, e.g., to tissue adjacent the inflamed tissue volume (e.g., above the inflamed volume), to the inflamed tissue volume itself, and/or to both the tissue adjacent the inflamed tissue volume and to the inflamed tissue volume itself. In another embodiment, the inflammation detector signals the user to deliver radiation due to the presence of the region of tissue containing inflammation. The repetition rate, specifically, the number of pulses of energy administered per unit of time that are delivered in a single scan can vary as a function of inflammation detected by the inflammation detector. Alternatively, or in addition, the frequency of energy scans administered can vary as a function of the inflammation detected by the inflammation detector.

Optionally, the device is wearable and is removably attachable to a subject's body. The wearable device can be, for example, one of a patch or a garment. The radiation can be electromagnetic radiation or the radiation can be one of optical radiation, ultrasound radiation, and radio frequency radiation. The device releases the radiation in a controlled manner, with pulse repetition rate approximately between 0.001 Hz and 10 Hz. In some embodiments, the device may monitor the status of the inflamed tissue (e.g., through temperature monitoring) and vary the release rate accordingly.

In another aspect, the disclosure relates to a wearable and removably attachable device for treating inflammation or pain, the device includes a source of radiation configured to generate radiation to damage a volume of tissue. The device has an optical path that delivers radiation from the source to the volume of tissue to form a damaged portion to a depth of at least about 50 microns, to a depth of at least about 100 microns, or to a depth of between about 50 microns and about 1000 microns of the volume where the damaged portion of the volume is separated from another damaged portion of the volume by an non-treated portion of the volume. The device also includes a controller for delivering the radiation at preprogrammed time intervals. Preprogrammed time intervals can include, for example, every 10 minutes, every 30 minutes, hourly, every 4 hours, every day, every other day etc. The wearable and removably attachable device may be, for example, a patch or a garment.

In certain embodiments, controlled temperature changes may be used for treatment of inflamed tissue by forming one or more damaged portions in inflamed tissue. Thermal treatments include cooling or cycled cooling and heating. A thermal element can provide cooling, heating, a combination of cooling and heating, or a cycled combination of cooling and heating. In one embodiment, the thermal element includes a plurality of cooling elements adjacent to a plurality of heating elements.

### BRIEF DESCRIPTION OF THE DRAWINGS

The following drawings are illustrative and are not meant to limit the scope of the disclosure.

FIG. 1 is a schematic view of various embodiments of treatment islets.

FIG. 2 is a schematic diagram showing EMR of a beam focused to a focal point.

FIGS. 3A and 3B are semi-schematic perspective and side views respectively of a section of a patient's skin and of equipment positioned thereon for practicing one embodiment of pain treatment.

FIG. 4 is a perspective view of an embodiment for creating treatment islets.

FIG. 5A is a bottom view of an embodiment for created treatment islets, which uses one or more capacitive imaging arrays.

FIG. 5B is a side view of an embodiment using a diode laser bar.

FIG. 6A shows a contact tip having multiple sub-regions (e.g., protrusions) having a square shape.

FIG. 6B shows a contact tip having multiple sub-regions (e.g., protrusions) having a rectangular shape.

FIG. 6C shows a contact tip having multiple sub-regions (e.g., protrusions) having a grooved shape.

FIG. 6D shows a fractional contact tip having a protrusion being pressed into a subject's skin.

FIG. 7 is a schematic perspective view of a wearable device for pain treatment.

FIG. 8 is a side view of the device of FIG. 7, showing a fractional radiation source and various other components of a wearable device.

FIG. 9 is a tabulation of results from a study that quantifies relief from “worst pain” and “average pain,” as reported by a live device group and a placebo group.

FIG. 10 is a graph of percentage of subjects experiencing pain relief versus time for the live device group and the placebo group. (This graph also provides separate data for “knee-pain only” subgroups of both the live device group and the placebo group.)

FIG. 11 is a graph of percentage of subjects experiencing pain relief versus time for the placebo group and the follow-up study in which the placebo group was given live devices.

FIG. 12 is a graph of duration of pain relief reported by subjects versus time for the live device group and the placebo group.

FIG. 13 is a graph similar to FIG. 12 but also including the follow-up study in which the placebo group was given live devices.

FIG. 14 is a photograph showing actual treatment sites on a subject.

FIG. 15 is an enlargement photograph showing an array of micro islets formed at a treatment site.

FIG. 16A is a cross-sectional microphotograph of a histological section slide showing the penetration depth of a single treat column with 15 mJ applied to the treated islet (wavelength 1540 nm).

FIG. 16B is a cross-sectional microphotograph of a histological section slide showing the penetration depth of a single treat column with 30 mJ applied to the treated islet (wavelength 1540 nm).

FIG. 16C is a cross-sectional microphotograph of a histological section slide showing the penetration depth of a single treat column with 50 mJ applied to the treated islet (wavelength 1540 nm).

FIG. 16D is a cross-sectional microphotograph of a histological section slide showing the penetration depth of a single treat column with 100 mJ applied to the treated islet (wavelength 1540 nm).

FIG. 17 is a graph showing experimentally measured dependence of the depth and diameter of the columns of non-ablative micro-damage on pulse energy for a wavelength of 1410 nm.

### DETAILED DESCRIPTION

The present disclosure describes fractional irradiation methods and devices that apply radiation to a multitude of points to create a multitude of damaged portions that are separated from one another by untreated portions and these irradiation methods and devices treat and/or control pain, pain associated with inflammation, and/or inflammation itself (including inflammation associated with pain). In accordance with the treatment methods lattices of radiation-treated islets are applied to the regions of tissue containing inflamed tissue. The radiation applied is suitable for damaging portions of the region of tissue containing inflammation, e.g., to tissue adjacent the inflamed volume (e.g., above the inflamed volume) and the radiation applied is also suitable for damaging portions of the volume of inflamed tissue such that a damaged portion of the inflamed tissue is separated from another damaged portion by a non-treated portion of the volume. The disclosed fractional treatment of inflammation, pain, and pain associated with inflammation is an alternative non-exogenous opioid treatment. Without being bound to any single theory it is believed that the use of the fractional device is a stress producer that stresses the region of tissue including the targeted inflamed tissue and as a result of the stress a process is instigated that delivers opiates to a region of tissue including the inflamed tissue and/or to the inflamed tissue itself, which reduces inflammation and/or reduces pain and/or pain associated with inflammation. The methods and devices disclosed herein are particularly effective after the inflammatory stage of wound healing is well established, for example, after at least 10 minutes of inflammation, after from about 30 minutes to about 24 hours of inflammation, or after about six hours of inflammation.

The subject's immune system recognizes the damage columns applied to the region of tissue including the volume of inflammation as a foreign body or as multiple foreign bodies and as a result the immune systems pain relief response is activated. The foreign bodies provide signals to the immune system that combine to tell the body how to respond. Treating the region of tissue including the inflamed volume with radiation to provide thermally damaged portions separated by non-treated portion(s) creates damage to the tissue volume portions that the body itself will resolve, clear up, and/or heal. One key to the inflicted damage is that the body can resolve and/or heal the damage without assistance. Without being bound to any single theory, it is believed that inflicting damaged portions on the region of tissue including the inflamed volume of tissue causes an acute injury that initiates a cascade of actions involving the Corticotropin Release Hormone, which serves to increase the delivery of endogenous opiates responsible for the disruption of pain, inflammation, and/or pain associated with the inflammation while the body works to resolve the damage present in the region of tissue including the inflamed portion of the volume of tissue. The perception of pain relief can last for all or a portion of the time that is required for the body to heal the damage. For example, the pain relief can last for all or a portion of the time that is required for the body to heal the fractional damage, which can take minutes and/or hours and/or days to achieve complete healing. An additional treatment (e.g., an additional application of damage columns applied to the region of tissue including a volume of inflammation) to relieve pain (e.g., chronic pain such as joint pain) may be required once the previously applied damage is healed and/or is substantially healed.

The devices and methods of the present disclosure include the application of directed energy and/or radiation which is applied in a multitude of points (e.g., fractionally) to a patient's tissue (e.g., skin) in a region where suppression of pain, the perception of pain, pain associated with inflammation, and/or inflammation (including inflammation associated with pain) is desired. In various embodiments, examples of which are described in greater detail below, devices and systems are provided for treating pain by producing lattices of radiation-treated islets (e.g., damaged portions) in target tissue regions.

In one embodiment, the directed energy is optical energy (e.g., light energy), which can be delivered with parameters summarized in Table 1A.

TABLE 1A
Pain and/or inflammation reduction treatment parameters
where the directed energy is light energy

| | | Energy per | Microbeam | Interval between | |
|---|---|---|---|---|---|
| Wavelength | Pulse width | microbeam, | density | consecutive | Interval between |
| (nm) | (ms) | (mJ) | (1/cm²) | pulses (sec) | applications (sec) |
| 250-12000 | 0.001-1000 | 0.01-1000 | 1-1000 | 0-10000 | 1-100000 |

In some embodiments, the wavelength range is from about 1000 nm to about 3000 nm and the energy is optical energy (e.g., light energy), delivered with parameters summarized in Table 1B.

TABLE 1B
Pain and/or inflammation reduction treatment parameters
where the directed energy is light energy

| | | Energy per | Microbeam | Interval between | |
|---|---|---|---|---|---|
| Wavelength | Pulse width | microbeam | density | consecutive | Interval between |
| (nm) | (ms) | (mJ) | (1/cm²) | pulses (sec) | applications (sec) |
| 1000-3000 | 1-1000 | 1-100 | 4-200 | 0.001-1 | 1000-50000 |

In some embodiments, the wavelength range is from about 1200 nm to about 1700 nm and the energy is optical energy (e.g., light energy), delivered with parameters summarized in Table 1C.

TABLE 1C
Pain and/or inflammation reduction treatment parameters
where the directed energy is light energy

| | | Energy per | Microbeam | Interval between | |
|---|---|---|---|---|---|
| Wavelength | Pulse width | microbeam | density | consecutive | Interval between |
| (nm) | (ms) | (mJ) | (1/cm²) | pulses (sec) | applications (sec) |
| 1200-1700 | 2-100 | 3-40 | 20-100 | 0.01-0.5 | 10000-50000 |

In some embodiments, the wavelength range is from about 1390 nm to about 1430 nm and the energy is optical energy (e.g., light energy), delivered with parameters summarized in Table 2.

TABLE 2
Pain and/or inflammation reduction treatment parameters
where the directed energy is light energy

| | Pulse | Energy per | Output power, | |
|---|---|---|---|---|
| Wavelength | width | microbeam | range | Interval between |
| (nm) | (ms) | (mJ) | (W) | applications (sec) |
| 1390-1430 | 4-20 | 6-30 | 1.35-1.65 | 1-100000 |

Tables 3A,B exemplify ranges of parameters for a focused ultrasound-based system.

TABLE 3A
Pain and/or inflammation reduction treatment parameters
where the directed energy is ultrasound energy

| | | Energy density | | |
|---|---|---|---|---|
| Frequency | Pulse width | in microbeam | Output power per | Numerical |
| (MHz) | (ms) | (J/cm²) | microbeam (W) | aperture |
| 0.1-50 | 1-500 | 1-100 | 0.1-100 | 0.5-1.2 |

TABLE 3B
Preferred pain and/or inflammation reduction treatment parameters
where the directed energy is ultrasound energy

| | | Energy density | | |
|---|---|---|---|---|
| Frequency | Pulse width | in microbeam | Output power per | Numerical |
| (MHz) | (ms) | (J/cm²) | microbeam (W) | aperture |
| 3-25 | 10-50 | 4-80 | 1-20 | 0.8-1.1 |

Tables 4A,B exemplify ranges of parameters for an radiofrequency-based (RF-based) system.

TABLE 4A
Pain and/or inflammation reduction treatment parameters
where the directed energy is RF energy

| | | | Voltage on high- | |
|---|---|---|---|---|
| Frequency | Pulse width | Energy per pulse | impedance load | Current |
| (MHz) | (ms) | (J) | (V) | (mA) |
| 0.1-10 | 1-500 | 0.1-10 | 500-1000 | 10-1000 |

TABLE 4B
Preferred pain and/or inflammation reduction treatment
parameters where the directed energy is RF energy

| | | | Voltage on high- | |
|---|---|---|---|---|
| Frequency | Pulse width | Energy per pulse | impedance load | Current |
| (MHz) | (ms) | (J) | (V) | (mA) |
| 0.5-5 | 20-150 | 0.5-4 | 700-900 | 50-400 |

The immediate biological effect of the application of fractionated directed energy on the tissue can range from mild stress (mechanical or thermal) to damage (including coagulative damage and/or ablative removal of a volume of tissue). While not being bound to any single theory of how pain reduction is enabled via the application of directed energy, and more particularly the application of fractionated directed energy, the following non-exclusive list of mechanisms that may be relevant to pain reduction include:
1) Extravasation. Variation of blood vessel permeability, facilitating passage of cellular blood components and blood plasma into the interstitial space of the blood vessel(s). This process may have a direct effect on inflammation effecting pain.
2) Modulation of transmission of pain signals through neurons by neurostimulation. This idea postulates an electrochemical process resulting in varying the neuron impedance. This may affect the process of pain signal transmission from a peripheral source to a regional plexus and, subsequently, to the brain. The interruption of transmission of pain signals can occur at various locations, e.g., Rolando's substantia gelatinosa, by overload or saturation of neuronal functions. This is the Melzack and Wall gate theory.
3) Stimulation of production of endogenous hormones suppressing pain (e.g., endorphins). This can occur through several intermediate pathways, either as a result of direct exposure of endorphin-producing centers to light or as a mediated response to peripheral exposure.
4) Controlling the Corticotropin Releasing Hormone (CRH) by stressing the tissue to release the CRH peptide to enhance the analgesic effect of the leukocyte.
5) Regions of damage. The denaturing tissue effect of the heat columns of tissue (coagulated tissue) can be considered “micro wounds.” The generation of these wounds elicits a wound healing cascade and during the inflammatory phase of the wound healing cascade there is de novo production of opioids.

A non-exclusive list of conditions that can be treated using the device and the method of the present disclosure includes: joint inflammation, chronic joint inflammation, joint pain, chronic joint pain, autoimmune caused joint inflammation, traumatic wound inflammation, postoperative inflammation, lower back pain, sciatica, neck pain, whiplash, facet syndrome, myofascial pain, trigger points, interstitial cystitis, degenerative joint disease of hands, knees, ankles, hips or feet, CTS, epicondylitis (lateral & medial), radiculitis. plantar fasciitis, biceps tendonitis, patellar tendonitis, hamstring tears, ankle sprains, medial collateral ligament strains, trochanteric bursitis, piriformis syndrome, arthroscopy related pain, AC joint sprains, ACL repair, shin splint, posterior tibialis tendonitis, rotator cuff tendonitis, hip flexor strains, fibromyalgia, intercostal neuritis, sacroilleitis, edema associated with soft tissue or joint trauma, TMJ pain, scar remodeling associated with surgical incisions, metatarsalgia, Morton's neuroma, ulnar neuritis, DeQuervain's tenosynovitis, wrist pain-unspecified, thoracic outlet syndrome, RSD reflex sympathetic dystrophy, muscle strain/spasm, phantom pain (e.g. the experience of pain perceived to be coming from a missing appendage and/or limb), and neurogenic migraine headaches.

When using radiation such as electromagnetic radiation (EMR), optical radiation, ultrasound radiation, and/or radiofrequency radiation and other forms of energy to treat tissues, there are substantial advantages to producing lattices of treated islets (e.g., damaged portions) in the tissue rather than large, continuous regions of treated tissue. The lattices are periodic patterns of treated islets in one, two or three dimensions in which the islets correspond to locally treated tissue. The islets are separated from each other by non-treated tissue (or differently- or less-treated tissue) such that a damaged portion of the volume of tissue is separated from another damaged portion by a non-treated portion of the volume.

In accordance with fractional treatment methodologies, the radiation treatment results in a lattice of treated islets which have been exposed to a particular wavelength or radiation spectrum (e.g., EMR spectrum), and which is referred to herein as a lattice of islets. When the absorption of radiation energy results in significant temperature elevation in the treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy is absorbed that is sufficient to denature and/or coagulate the lattice is referred to herein as a lattice of “photochemical islets.” When an amount of energy is absorbed that is sufficient to ablate the tissue being treated, the lattice is referred to herein as a lattice of “ablated islets” or “ablation islets.” When the islets are sufficiently small, for example, on the order of approximately 2 mm or less, the islets can also be referred to herein as a lattice of “micro-islets.” Micro-islets can be various sizes, including, without limitation, micro-islets that are macroscopic or microscopic in size. Additionally, the orientation of the islets can be varied from normal to a tissue surface, to parallel with the surface, or at other angles or orientations, including islets that are curved or otherwise are not formed along a straight path.

An extensive discussion of the various types of treated islets (such as damage islets, thermal islets, photochemical islets and ablated islets) as well as the parameters and specification of devices used to form such types of islets during a fractional treatment can be found in U.S. Pat. No. 6,997,923 entitled “Method and Apparatus for EMR Treatment,” United States Publication No. 2004/0147984 entitled “Method and Apparatus for Delivering Low Power Optical Treatments,” United States Publication No. 2006/0058712 entitled “Methods and Products for Producing Lattices of EMR-Treated Islets in Tissues, and Uses Therefore,” United States Publication No. 2008/0058783 entitled “Handheld Photocosmetic Device,” United States Publication No. 2006/0058712 entitled “Use Of Fractional EMR Technology On Incisions And Internal Tissues,” United States Publication No. 2008/0172047 entitled “Methods and Devices for Fractional Ablation of Tissue,” United States Publication No. 2009/0069741 entitled “Methods and Devices for Fractional Ablation of Tissue For Substance Delivery,” United States Publication No. 2008/0186591 entitled “Dermatological Device Having A Zoom Lens System,” United States Publication No. 2008/0294150 entitled “Photoselective Islets In Skin and Other Tissues,” United States Publication No. 2009/0254076 entitled “Method and Apparatus for Fractional Deformation and Treatment of Tissue,” United States Publication No. 2010/0036295 entitled “Method and Apparatus for Fractional Deformation and Treatment of Cutaneous and Subcutaneous Tissue,” United States Publication No. 2010/0298744 entitled “System and Method of Treating Tissue with Ultrasound Energy,” and United States Publication No. 2010/0286673 entitled “Method and Apparatus for Treatment of Tissue,” and the disclosure in these patents, patent applications and their family members are incorporated by reference herein.

In some embodiments, an islet is a small treated volume (V_{eff}) in the tissue in which the tissue has been damaged, ablated or otherwise treated to form small hot spots, holes, channels, grooves, openings, chambers and/or similar structures in the tissue.

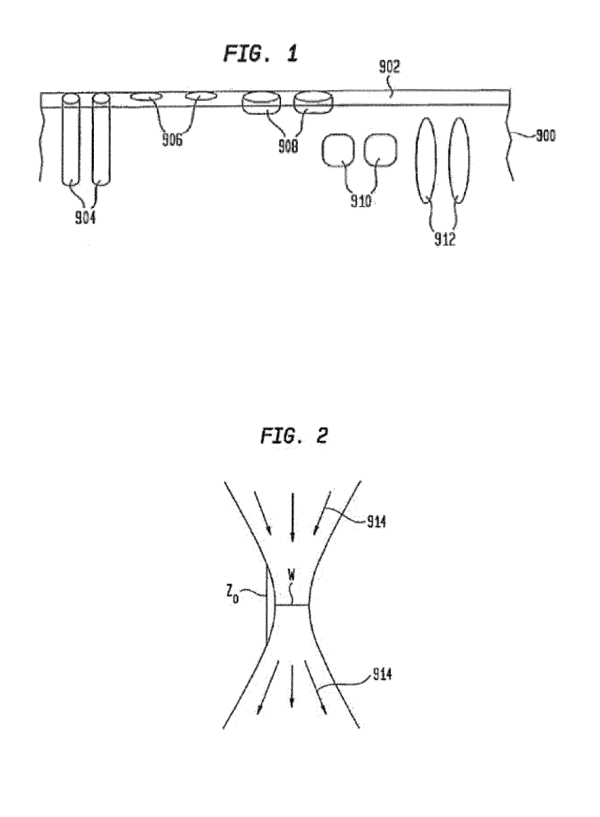

Referring to FIG. 1, examples of various micro-islet structures are shown. Micro-islet structures may be formed by non-ablative and/or by ablative means. The treated volumes 904 are columns extending from a surface 902 of tissue 900 and into the tissue 900 (e.g., into a depth of the tissue 900). Treatment volumes 906 lie at the surface 902 of the tissue, but that do not extend deeply into the tissue. Treatment volumes 908 lie at the surface 902, but that extend slightly into the tissue 900 to a greater depth than treatment volumes 906. The treatment volumes 904 lie at the surface 902, but that extend into the tissue 900 to a greater depth than treatment volumes 908. Treatment volumes 910 are chambers within the tissue 900 and below the surface 902. Similarly, treatment volumes 912 are chambers that are elongated to form columns but that do not have an opening through the surface. The treatment volumes shown in FIG. 1 are simplified to aid in description.

Depending on how a treatment volume is formed, its structure may be more complex. For example, a micro-hole formed by ablating tissue may have a zone or halo of coagulated damage surrounding the vacated hole.

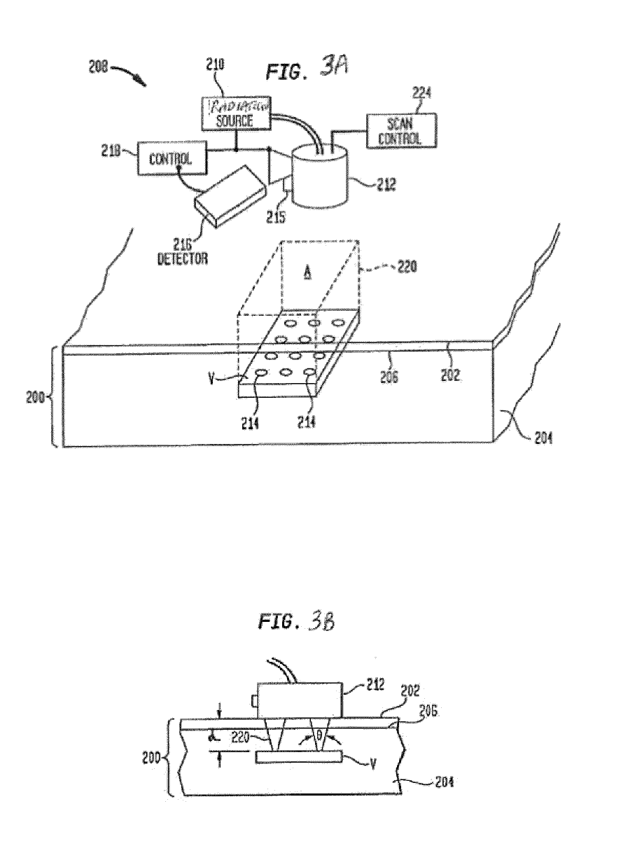

Referring to FIG. 2, in one embodiment, the size of a treatment volume is determined essentially by the spot size at which radiation is applied to the tissue and the power density of the radiation that is applied. In some embodiments, the radiation is EMR and the wavelength of EMR that is applied and the threshold of treatment of the tissue that is irradiated (for example, the threshold of thermal damage or the threshold of ablation) also determine the treatment volume. To maximize the intensity of the radiation, the spot size of a micro-islet is preferably the diameter of the focal point. Using currently available optics, therefore, treatment islets can be formed having a diameter of approximately 0.1×λ (i.e., 10% of the wavelength of the applied radiation). However, even smaller diameters are theoretically possible, depending on the quality of optics and the design of optics that are used.

The spot size that can be created (and, thus, the resulting micro-islet) is proportional to the wavelength: the smaller the wavelength, the smaller the micro-islet that can be created. FIG. 2 shows a focused beam of rays 914 of EMR in which the focal point has a diameter W greater than the wavelength of the EMR. Theoretically, the smallest spot size that is possible for an individual EMR beam is the smallest focal point that can be achieved. The smallest focal point that may be achieved has a diameter (W) that is approximately the wavelength (λ) of the EMR that is applied. (Although the term focal point is used, one skilled in the art will understand that light does not focus to a point and instead has an area with a diameter that is typically referred to as the waist of the beam.)

If non-coherent light is applied, the smallest spot size that is theoretically possible is the largest wavelength among the wavelengths that are applied to achieve a treatment effect on the tissue, such as a thermally damaged (e.g., coagulated) micro-islet. This would not include longer wavelengths that do not have an effect that forms an EMR-treatment islet. For example, if one or more spectral bands of EMR are applied to the tissue, but only a subset, subsets, or sub-band(s) of the EMR are actually used to treat and form the islet, the smallest possible diameter of the resulting micro-islet will be the size of the largest wavelength in the sub-band(s) or subset(s) of EMR.

Because smaller focal areas are possible using shorter wavelengths, one effective means for creating very small micro-islets is the use of an excimer laser or another laser to produce EMR in the ultraviolet range.

The focal depth (Z₀) of the spot size is a function of the diameter of the focal point, which is determined by the following equation:

$${Z}_{0} = \frac{π * {W}^{2}}{λ} \qquad ( 1 )$$

Thus, in an example where the focal point has a diameter of 30 μm and the wavelength is 3 μm, the focal depth is approximately 943 μm.

Although embodiments disclosed herein include optical systems and/or elements to focus radiation (e.g., EMR) at a focal point, such focusing is not necessarily required to practice all of the embodiments taught by the disclosure.

In other embodiments, the power density may be modulated during the formation of a single micro-islet. For example, a first pulse of EMR can be applied at a first power density and a second pulse can be applied at a different power density. If the power densities of multiple pulses are alternated in this fashion, micro-islets having varying diameters can be formed. Such micro-islets may have various benefits, for example the shape of the micro-column produced in such a way can be optimized for a particular pain and/or inflammation condition. Similarly, the power density can be modulated, for example, between pulses, during pulses or during the application of EMR in a continuous or quasi-continuous wave, to form micro-islets of varying shapes, such as, for example a conical-like shape. A conical shape in which the narrow portion of the cone is at the surface of the tissue and in which the wider base of the cone lays within the tissue could be used to create a treated volume having a relatively larger volume. In another embodiment, the micro-islet has a columnar shape.

Micro-islets may be disposed at the surface of the tissue or may extend at a depth into the tissue including relatively deeply into the tissue, for example, from the surface of the skin into muscle tissue, from the surface of the skin into a joint, from the surface of the skin to a depth of at least 50 microns, from the surface of the skin to a depth of from about 50 microns to about 1000 microns. There are several mechanisms available to create relatively deep micro-structures. For example, a device may have one or more of the following features: an optical system designed for irradiating tissue below the surface; a mechanism to adjust the focus deeper into the tissue as the micro-structure is formed; a high-aspect ratio; and a relatively longer focal length. Another mechanism is mechanical stretching of the skin to decrease density and increase depth of penetration (e.g., use of a point compression array such the Palomar® XD Microlens™, which aids in access to a depth of non-ablative fractional treatment(s)).

In some embodiments, repeated pulsing can be advantageous in forming treatment volumes. However, when a single pulse of radiation is applied in a system, for example, aligned such that a focal area of the radiation (e.g., EMR) just below a tissue surface, multiple pulses of energy will gradually have less intensity deeper in the tissue as the beam diverges (as shown in FIG. 2). Thus, if such a system is used to create the treated volumes that extend more deeply, additional mechanisms may be used in conjunction with multiple pulses.

The treated volumes may also take on many shapes and patterns such as, for example, arrays of elongated islets including arrays of straight rows, parallel rows, regularly-spaced curved rows, or intersecting rows. The treated volumes may be V-shaped or may have many different alternative configurations, including, without limitation, a U-shaped trough, a circular-shaped trough, a rectangular shape, a cross-section that is wider at the base than at the top, or a relatively narrow neck with a larger treated volume below the tissue surface.

Furthermore, the treated volumes can be formed by a number of different mechanisms. For example, an elongated islet of damage can be formed by a single beam continuously scanned along a path. The islets can be formed using a phase array. A cylindrical lens or similar lens may be used to focus radiation along a path on the tissue where the elongated islet will be formed. Additionally, a set of pulses of radiation may be generated either sequentially or simultaneously to form a set of spots on or in the tissue. When tissue is treated at the spots, the cumulative result is a single elongated islet or a set of elongated islets. In still other embodiments, the treated volume may be circles, semicircles, and concentric circles. Additionally, combinations of grooves and other micro-structures or types of EMR-treated islets (both ablative and non-ablative) can be used, such as micro-holes in combination with a circular micro-groove or an elongated damage EMR-treated islet in between concentric circles or in between intersecting grooves. Many other embodiments are possible, including other shapes, patterns, dimensions, and combinations.

In a given lattice of treated islets, the percentage of tissue volume which is treated is referred to as the “fill factor” or f. The fill factor is defined by the volume of the islets with respect to a reference volume that contains all of the islets. The fill factor may be uniform for a periodic lattice of uniformly sized treated islets, or it may vary over the treatment area. Non-uniform fill factors may desirably be created. For such situations, an average fill factor (f_{avg}) can be calculated by dividing the volume of all treated islets Vᵢ^{islet} by the volume of all tissue Vᵢ^{tissue} in the treatment region,

$${f}_{avg} = {Σ}_{i} \frac{{V}_{i}^{islet}}{{V}_{i}^{tissue}} . \qquad ( 2 )$$

Generally, the fill factor can be decreased by increasing the center-to-center distance(s) of islets of fixed volume(s), and/or decreasing the volume(s) of islets of fixed center-to-center distance(s). Thus, the calculation of the fill factor will depend on volume of an EMR-treated islet as well as on the spacing between the islets. In a periodic lattice, where the centers of the nearest islets are separated by a distance d, the fill factor will depend on the ratio of the size of the islet to the spacing between the nearest islets d. For example, in a lattice of parallel cylindrical islets, the fill factor will be:

$$f = {π ( \frac{r}{d} )}^{2} , \qquad ( 3 )$$

where d is the shortest distance between the centers of the nearest islets and r is the radius of a cylindrical EMR-treated islet. In a lattice of spherical islets, the fill factor will be the ratio of the volume of the spherical islet to the volume of the cube defined by the neighboring centers of the islets:

$$f = \frac{4 π}{3} {( \frac{r}{d} )}^{3} , \qquad ( 4 )$$

where d is the shortest distance between the centers of the nearest islets and r is the radius of a spherical EMR-treated islet. Similar formulas can be obtained to calculate fill factors of lattices of islets of different shapes, such as lines, disks, ellipsoids, rectangular areas, or other shapes.

The center-to-center spacing (i.e., pitch) of islets is determined by a number of factors, including the size of the islets and the treatment being performed. Generally, it is desired that the spacing between adjacent islets be sufficient to protect the tissues and facilitate the healing of any damage thereto, while still permitting the desired therapeutic effect to be achieved. In general, the fill factor can vary in the range of 0.01-90%, with ranges of 0.1-1%, 0.1%-10%, 1-10%, 10-30% and 30-50% for different applications.

In some embodiments producing thermal islets, the fill factor may be sufficiently low to prevent excessive heating and damage to islets. In some embodiments producing damage islets, the fill factor may be sufficiently low to ensure that there is undamaged tissue around each of the damage islets sufficient to prevent bulk tissue damage and to permit the damaged volumes to heal. The specific parameters, such as the degree of separation and the ratio of the volume of islets to the volume of tissue that is treated but in which islets are not formed, will vary depending on the application. In some embodiments, for example, the entire treated tissue could be irradiated to some degree, such as to cause a thermal reaction in the tissue or a degree of damage in the tissue while the EMR-treated islets would be formed within that tissue and would have a greater degree of damage. For example, a lattice of damage islets could be formed within a volume of tissue that has been treated to provide an underlying bias of heat throughout the volume of tissue.

Suitable devices can create damage columns (e.g., damage islets) and/or ablated holes in tissue with great flexibility. For example, suitable devices can precisely space the damage columns and/or ablated holes, can control the depth of the columns and/or the depth of the ablated holes, can control the rate at which the damage columns and/or ablated holes are created and/or can control the number of damage columns in a region. In this way, the device can be tailored for pain control and to provide stress induced analgesia. It is postulated that stress related hormonal activity plays an essential role in the endogenous control of pain. Thus, control of stress inflicted on the tissue is essential to endogenous pain control. The stress inflicted on the tissue can be controlled by, for example, control of the density of damage sites (i.e., the density of the stress sites), as well depth, diameter, and shape of individual columns and/or channels. In some embodiments, the device delivers treatment to a relatively compact region (e.g., the treatment area is small). The efficacy of the treatment can depend on how precisely the treatment is conducted in as limited a volumetric area as you can define. The improvement and/or reduction of pain perception can be dependent upon the volume of the stress that is applied to the tissue. In a fractional technique the intensity of the treatment (e.g., the density of the damage sites) can be more important than the overall volume over which the effect is distributed. In one embodiment, the device is capable of delivering from about 1 beam to several beam fractions (e.g., 100). The device can provide from about 1 beam per cm² to about 100 beams per cm². Optionally, the size of each beam is variable. In this way, each beam can have a unique intensity and the number of beams and their size can be targeted to a specific application. In some embodiments, fractional treatment technology provides control via a repetitive action.

Where the treatment goal is to accentuate interruption of the pain mechanism relative to inducing opiate production then one approach is to decrease the applied power while having a maximum spot density to decrease the perceived pain. Where the goal is to accentuate opiate production relative to interruption and to decrease the transmission of pain and/or improve pain management one can change the nature of the damage by (a) increasing the power, (b) increasing the damage island density and/or (c) increasing the footprint (e.g., by sliding or gliding the device rather than stamping the device).

In some embodiments, the device is a wearable device (e.g., a garment) that may be particularly well suited to chronic pain and/or chronic inflammation. For example, the wearable device may be well suited to chronic joint pain. Suitable wearable devices are discussed in greater detail below.

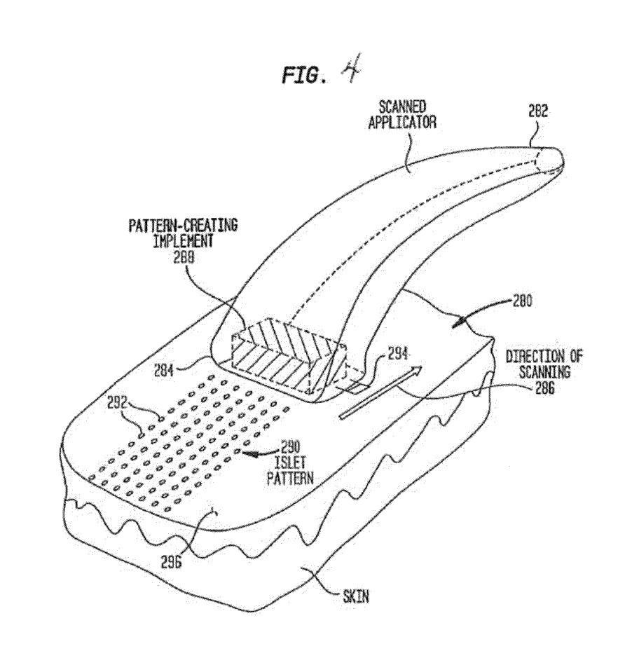

Referring to FIGS. 3A and 3B, each of the treated volumes can be a relatively thin disk, a relatively elongated cylinder (e.g., extending from a first depth to a second depth), or a substantially linear volume having a length which substantially exceeds its width and depth, and which is oriented substantially parallel to the skin surface. The islets can also be substantially linear or planar volumes. The type of islet and the orientation of the islets 214 in a given application need not all be the same. For example, where the islets are substantially linear, some of the lines may, for example, be at right angles to other lines. Lines also can be oriented around a treatment target for greater efficacy.

Treated islets 214 can be subsurface volumes, such as spheres, ellipsoids, cubes or rectangular volumes of selected thickness. The shapes of the islets are determined by the combined optical parameters of the beam, including beam size, amplitude and phase distribution, the duration of application and, to a lesser extent, the wavelength. The parameters for obtaining a particular islet shape can be determined empirically with only routine experimentation.

The described devices can be employed in the treatment of pain, inflammation, and inflammation associated with pain. The area of the body to be treated is skin 200, which is a region of tissue that includes a volume of inflamed tissue. The volume of inflamed tissue may be located at a depth in the tissue volume (e.g., at the depth of the muscle or the joint) or, alternatively, it may be present at the epidermis. In order for the peripheral opiate analgesia to be enabled, encouraged, facilitated, and promoted by the methods and devices disclosed herein the inflammation has preferably already taken hold in the inflamed volume prior to treatment of the region of skin 200 with the device to cause damaged portions of the tissue volume separated by non-damaged or lesser damaged portions of the tissue volume. In some embodiments, inflammation has been allowed to take hold in the inflamed volume of tissue, for example, inflammation has been established for at least about 10 minutes, or from about 30 minutes to about 6 hours, for example. Endogenous opioids are provided to the inflamed injury when damage is provided by the applied radiation; the endogenous opioids are likely to travel to the peripheral nervous system rather than the central nervous system, which is desirable. We believe the by providing fractional injury and/or damage to the tissue region containing a volume of established inflammation, the body and/or it's immune system is signaled that the multiple portions of damage are foreign bodies being introduced to the inflammation site. In this way the process of providing peripheral opioid analgesia can be multiplied and/or increased and can provide analgesia that results in improved inflammation, treatment of pain, and treatment of pain associated with inflammation.

Inflamed tissue is targeted for treatment in part because in the inflammation phase the peripheral nerve system receptors are more prevalent than the central nerve system receptors. It is desirable that the peripheral nerve system receptors receive the endogenous opioids rather than the central nervous system receptors. Thus, it is important that inflammation on the body of the subject be detected. One or more of the presence of inflammation, its location, the region and/or location on the body of the subject, the depth of inflammation in the body of the subject (e.g., at a depth in the tissue of the subject), the severity, and/or the level of inflammation should be determined. In some embodiments, the sufferer of the pain is part of the inflammation detection (e.g., the sufferer of the pain determines if it is muscle pain, nerve pain, shallow pain, and/or deep pain etc.). For example, in one embodiment, the volume of inflamed tissue is determined by the subject, i.e., the sufferer of the pain feeling that the location is tender to the touch. In another embodiment, the volume of inflamed tissue is determined by the subject feeling that the location feels relatively warmer in temperature than other regions of tissue.

The effectiveness of this treatment method can be impacted by the condition of the subject's immune system, for example, it is preferable that the subject has a healthy and/or well-functioning immune system that is capable of recognizing a foreign body or foreign bodies. Thus, a cancer patient with an impaired immune system would not benefit, as well, from this approach to pain and inflammation as would a subject with an otherwise healthy immune system. For example, a multiple myeloma patient who has arthritis type of pain would be unlikely to benefit from this treatment, or would likely not benefit to the extent a subject with a healthy immune system who has arthritis would benefit, because the cancer patient's immune system would not react to the treatment as a foreign body since it is compromised.

A subject's immune system is triggered on by macrophage, which is a primary detector of problems or issues faced by the immune system. The macrophage is present in all tissues of the body and is on the lookout for problems or issues. Therefore, if a foreign body is introduced the macrophage signals action on the part of the immune system. In a person having a compromised immune system the quantity of macrophage present is reduced making the immune system less effective.

CRH is naturally present in the human body. CRH is a way of releasing peptides from the leukocytes and providing peripheral analgesia in the body. For the immune system to respond you need CRH to be present and you need inflammation to be present for effective treatment. There may be opportunity to increase the level of CRH in the body of the subject by, for example, drug delivery to the site of inflammation (e.g., topical drug delivery or injection to the site) or by regular CRH drug dosage (e.g., a regular oral regimen of a CRH supplement). The theory is if the amount of CRH available in a person's system is overall improved and/or increased when the CRH is attracted to foreign bodies applied to area of injury the quantity of CRH that is available in the body as a result of the oral or transcutaneous regimen is overall increased and thus the pain control capability is increased. Thus, a regular regimen of CRH (e.g., oral or transcutaneous) can increase the body's ability to manage pain and/or inflammation.

In one embodiment, an ablated shaped micro-hole is used to hold a substance, such as a topical for pain treatment (e.g., a topical containing CRH). When using ablation to form a micro-hole, the ablation is preferably performed in conjunction with a device to remove the ablated material, although this is not required. When tissue is ablated, remnants of the tissue can remain in the micro-holes. This can increase the amount of refraction and otherwise decrease optimum performance of the device forming the micro-holes. The micro-holes are formed more precisely when the ablated material is removed. In one embodiment, a device is synchronized to produce a short pulse of air at high pressure to expel the ablated material immediately after a pulse of EMR is applied before the ablated material has a chance to settle in the micro-hole that is being formed. Thus, ablated micro-holes may be employed to introduce a quantity of topical CRH to an area of inflammation (e.g., ablated micro-holes act as an avenue for topical introduction to the body).

Providing fractional treatment (e.g., injury and/or damage) to a region that includes the inflamed tissue is also helpful in that by creating a non-ablative fractional column with the radiation source the coagulated tissue of the non-ablative column acts a filter that masks some of the power of the radiation energy (e.g., the light source) such that a low level of light comes out of the bottom of the newly formed fractional non-ablative column. The exiting light provides a side benefit to the area of inflammation in that is provides a low level of light treatment therapy. For example where a 1440 nm wavelength is provided at a fluence of 15 mJ per dose to create a non-ablative coagulated column, the light source the exits the non-ablative coagulated column ranges between 1-5 mJ per dose. Thus, the power level is cut from down to measure from ⅓ to about 1/20 of the fluence that is provided by the fractional treatment device.

FIGS. 3A and 3B provide a schematic representation of a system 208 for creating islets of treatment (e.g., a damaged portion of a volume separated from another damaged portion of a volume by a non-treated portion of the volume). The system 208 is for delivering radiation to a treatment volume V located at a depth d in the patient's skin and having an area A. FIGS. 3A and 3B also show treatment or target portions 214 (i.e., islets of treatment) in the patient's skin 200. A portion of a patient's skin 200 is shown, which portion includes an epidermis 202 overlying a dermis 204, the junction of the epidermis and dermis being referred to as the dermis-epidermis (DE) junction 206. The treatment volume may be at the surface of the patient's skin (i.e., d is approximately equal to 0) such that islets of treatment are formed in the stratum corneum. In addition, the treatment volume V may be below the skin surface in one or more skin layers or the treatment volume may extend from the skin surface through one or more skin layers. In some embodiments, the treatment reaches to a depth of at least 20 microns, at least 50 microns, from about 20 to about 300 microns, or from about 50 to about 250 microns, or from about 20 microns to about 1000 microns.

In one exemplary embodiment, the inflamed tissue volume is at a depth of from about 100 microns to about 150 microns and the treatment radiation reaches to a depth of least 50 microns. In this instance the treatment method to treat the pain associated with the inflamed tissue treats a region that includes portions of tissue volume adjacent the inflamed tissue (e.g., from about 50 microns to about 100 microns) and the volume of inflamed tissue (e.g., from about 100 microns to about 150 microns).

In another exemplary embodiment, the inflamed tissue volume is at a depth of from about 125 microns to about 175 microns and the treatment radiation reaches to a depth of from about 50 microns to about 100 microns. In this instance, the treatment method to treat the pain associated with the inflamed tissue treats a region that includes portions of tissue volume adjacent the inflamed tissue (e.g., from about 50 microns to about 100 microns).

The system 208 of FIGS. 3A and 3B can be incorporated within a hand held device. The system 208 can include an energy source 210 more specifically, a radiation source such as, for example, an electromagnetic radiation (EMR) source, or a source of optical radiation, ultrasound radiation, or radio frequency radiation. In one embodiment, the output from the radiation source 210 is applied to an optical system 212, which is in the form of a delivery head that is in contact with the surface of the patient's skin, as shown in FIG. 3B. The delivery head can optionally include, for example, a contact plate or cooling element that contacts the patient's skin. Throughout this specification, the terms “head”, “hand piece” and “hand held device” may be used interchangeably.

Some embodiments can also include speed sensors, contact sensors, imaging arrays, and controllers to aid in various functions of applying radiation to the patient's skin. The system 208 can also include detectors 216 and controllers 218. The detectors 216 can, for instance, detect contact with the skin and/or the speed of movement of the device over the patient's skin and can, for example, image the patient's skin. The detector 216, may be, for example, a capacitive imaging array, a CCD camera, a photodetector, or other suitable detector for a selected characteristic of the patient's skin, such as inflammation. The output from detector 216 can be applied to a controller 218, which can be a suitably programmed microprocessor or other such circuitry, but may be special purpose hardware or a hybrid of hardware and software. The controller 218 can be used, for example, to control the radiation source as a function of its contact with the skin and/or the speed of movement of the hand piece.

Suitable detectors 216 can include inflammation detectors that detect inflammation in a region and/or a volume of tissue (e.g., can detect a hot spot of inflammation). For example, in some embodiments, such inflammation detectors 216 can determine the location of a volume of inflamed tissue (e.g., a volume of inflamed muscle tissue located at the depth of the inflamed muscle). Suitable inflammation detectors can be at least one of a thermometer, a medical IR thermal camera (available from FLIR Systems, MA), a thermally sensitive film (available from Sensor Products Inc, NJ), a video camera (available from Sony, Japan) and an ultrasound inflammation detector. Ultrasound can be employed to detect inflammation, because when inflammation occurs the body reacts by delivering cells from the immune system including leukocytes. Normally, these cells are delivered through the blood via the wall of the blood vessel, which becomes porous such that the cells of the immune system come out through the pores. As a side effect of the cell delivery bubbles come out of the pores and this is a sign of outgassing. The wavelength of the ultrasound detects the bubbles that indicate inflammation is present.

Controller 218 can, for example, control the turning on and turning off of the light source 210 or other mechanism for exposing the radiation (e.g., light) to the skin (e.g., a shutter) the control 218 may also control the power profile of the radiation. Controller 218 can also be used, for example, to control the focus depth for the optical system 212 and to control the portion or portions 214 to which radiation is focused/concentrated at any given time. Finally, controller 218 can be used to control the cooling element 215 to control both the skin temperature above the volume V and the cooling duration, both for pre-cooling and during irradiation.

In some embodiments, the detector(s) 216 detect the presence of a volume of inflamed tissue and as a result of the detected inflammation the controller(s) 218 control the radiation source thereby enabling it to perform a treatment due to the detection of inflammation. Thus, in one embodiment, the device operates only where inflammation is detected by the detector 216. In one embodiment, the detector 216 is a thermometer that detects inflammation. In other embodiments, the radiation source is enabled to perform when the detector 216 detects contact, but in addition, when the detector 216 detects the presence of inflamed tissue the user is provided with a signal regarding the presence of inflamed tissue in contact with the delivery head. Thus, the device could operate where inflammation is detected or in other areas but signals the user when to fire based on the detected inflammation. Suitable signals can include, for example, a visual signal such as a flashing light, an audible signal such as sound including, for example, a bell or a beep, or a sensed signal such as a vibration in the hand held device that indicates the delivery head is in contact with inflamed tissue. Optionally, where the device can operate in areas of inflammation and in other areas, the device can provide a level of treatment suitable for pain treatment only in areas (e.g., at locations on the subject's body) where the presence of inflammation in an inflamed tissue volume is detected by the detector 216 whereas the other non-inflamed tissues are provided with another level of treatment that is suitable for, for example, cosmetic treatment(s).

The radiation source 210 may be any suitable energy source, for example, EMR, ultrasound, radio-frequency, and optical radiation. Suitable optical radiation sources can include both coherent and non-coherent sources of optical radiation that are able to produce optical energy at a desired wavelength or a desired wavelength band or of multiple wavelengths or in multiple wavelength bands.

The energy source 210, and the energy chosen, may be a function of the type of treatment to be performed, the tissue to be heated, the depth within the tissue at which treatment is desired, and of the absorption of that energy in the desired area to be treated. For example, the radiation source 210 may be an optical energy source such as, for example, a radiant lamp, a halogen lamp, an incandescent lamp, an arc lamp, a fluorescent lamp, a light emitting diode, a laser (including diode and fiber lasers), or other suitable optical energy source. In addition, multiple energy sources may be used which are identical or different. For example, multiple laser sources may be used and they may generate optical energy having the same wavelength or different wavelengths. As another example, multiple lamp sources may be used and they may be filtered to provide the same or different wavelength band or bands. In addition, different types of sources may be included in the same device, for example, mixing both lasers and lamps. Energy source 210 may produce electromagnetic radiation, such as near infrared or visible light radiation over a broad spectrum, over a limited spectrum, or at a single wavelength, such as would be produced by a light emitting diode or a laser. In certain cases, a narrow spectral source may be preferable, as the wavelength(s) produced by the energy source may be targeted towards a specific tissue type or may be adapted for reaching a selected depth. In other embodiments, a wide spectral source may be preferable, for example, in systems where the wavelength(s) to be applied to the tissue may change, for example, by applying different filters, depending on the application. The radiation source 210 may be a source of ultrasound radiation, a source of radio frequency radiation, or another source may also be employed in suitable applications. In one embodiment, the radiation source in one device includes more than one source for example, a source of ultrasound radiation and laser radiation.

Lasers and other coherent light sources can be used to cover wavelengths within the 100 to 100,000 nm range. This includes wavelengths that are in wavelength ranges typically used for non-ablative procedures such as 1320 nm, 1450 nm and 1540 nm. Examples of coherent energy sources are solid state, dye, fiber, and other types of lasers. For example, a solid state laser with lamp or diode pumping can be used. The wavelength generated by such a laser can be in the range of 400-3,500 nm. This range can be extended to 100-20,000 nm by using non-linear frequency converting. One such laser is a 3 μm Erbium laser. Solid state lasers can provide maximum flexibility with pulse width range from femtoseconds to a continuous wave, preferably in a range of approximately 1 femtosecond to 100 milliseconds. When very short pulses of EMR are used to create micro-islets, the wavelength has a smaller effect. For example, when a pulse on the order of several femtoseconds is applied, the relationship between the wavelength and the focal area is less pronounced such that longer wavelengths may be used to create small structures.

Another example of a coherent source is a tunable laser. For example, a dye laser with non-coherent or coherent pumping, which provide wavelength-tunable light emission. Dye lasers can utilize a dye dissolved either in liquid or solid matrices. Typical tunable wavelength bands cover 400-1,200 nm and a laser bandwidth of about 0.1-10 nm. Mixtures of different dyes can provide multi wavelength emission. Dye laser conversion efficiency is about 0.1-1% for non-coherent pumping and up to about 80% with coherent pumping. Laser emission could be delivered to the treatment site by an optical waveguide, or, in other embodiments, a plurality of waveguides or laser media could be pumped by a plurality of laser sources (lamps) next to the treatment site. Such dye lasers can result in energy exposure up to several hundreds of J/cm², pulse duration from picoseconds to tens of seconds, and a fill factor from about 0.01% to 90%, from about 0.1% to about 50%.

One suitable coherent source is a fiber laser; fiber lasers are active waveguides having a doped core or undoped core (e.g., a Raman laser), with coherent or non-coherent pumping. Rare earth metal ions can be used as the doping material. The core and cladding materials can be quartz, glass or ceramic. The core diameter could be from one or more microns to hundreds of microns. Pumping light could be launched into the core through the core facet or through cladding. The light conversion efficiency of such a fiber laser could be up to about 80% and the wavelength range can be from about 1,100 to 3,000 nm. A combination of different rare-earth ions, with or without additional Raman conversion, can provide simultaneous generation of different wavelengths, which could benefit treatment results. The range can be extended with the help of second harmonic generation (SHG) or optical parametric oscillator (OPO) optically connected to the fiber laser output. Fiber lasers can be combined into the bundle or can be used as a single fiber laser. The optical output can be directed to the target with the help of a variety of optical elements described below, or can be directly placed in contact with the skin with or without a protective/cooling interface window. Such fiber lasers can result in energy exposures of up to about several hundreds of J/cm² and pulse durations from about picoseconds to tens of seconds.

Diode lasers can be used for the 400-100,000 nm range. In some embodiments, the configurations using diode laser bars can be based upon about 10-100 W, 1-cm-long, CW diode laser bar. Note that other sources (e.g., LEDs and microlasers) can be substituted in the configurations described for use with diode laser bars with suitable modifications to the optical and mechanical sub-systems. Other types of lasers (e.g., gas, excimer, etc.) can also be used.

A variety of non-coherent sources of electromagnetic radiation (e.g., arc lamps, incandescence lamps, halogen lamps, light bulbs) can be used for the energy source 210. There are several monochromatic lamps available such as, for example, hollow cathode lamps (HCL) and electrodeless discharge lamps (EDL). HCL and EDL could generate emission lines from chemical elements. For example, sodium emits bright yellow light at 550 nm. The output emission could be concentrated on the target with reflectors and concentrators. Energy exposures up to about several tens of J/cm², pulse durations from about picoseconds to tens of seconds, and fill factors of about 0.01% to 90%, from about 1% to 90%, or from about 0.1% to about 50% can be achieved.

Linear arc lamps use a plasma of noble gases overheated by pulsed electrical discharge as a light source. Commonly used gases are xenon, krypton and their mixtures, in different proportions. The filling pressure can be from about several torr to thousands of ton. The lamp envelope for the linear flash lamp can be made from fused silica, doped silica or glass, or sapphire. The emission bandwidth is about 180-2,500 nm for clear envelope, and could be modified with a proper choice of dopant ions inside the lamp envelope, dielectric coatings on the lamp envelope, absorptive filters, fluorescent converters, or a suitable combination of these approaches.

In some embodiments, a xenon-filled linear flash lamp with a trapezoidal concentrator made from BK7 glass can be used. The distal end of the optical train can, for example, be an array of micro-prisms attached to the output face of the concentrator. The spectral range of EMR generated by such a lamp can be about 300-2000 nm, energy exposure can be up to about 1,000 J/cm², and the pulse duration can be from about 0.1 ms to about 10 seconds.

Incandescent lamps are one of the most common light sources and have an emission band from 300 to 4,000 nm at a filament temperature of about 2,500° C. The output emission can be concentrated on the target with reflectors and/or concentrators. Incandescent lamps can achieve energy exposures of up to about several hundreds of J/cm² and pulse durations from about seconds to tens of seconds.

Halogen tungsten lamps utilize the halogen cycle to extend the lifetime of the lamp and permit it to operate at an elevated filament temperature (up to about 3,500° C.), which greatly improves optical output. The emission band of such a lamp is in the range of about 300 to 3,000 nm. The output emission can be concentrated on the target with reflectors and/or concentrators. Such lamps can achieve energy exposures of up to thousands of J/cm² and pulse durations from about 0.2 seconds to continuous emission.

Light-emitting diodes (LEDs) that emit light in the 290-2,000 nm range can be used to cover wavelengths not directly accessible by diode lasers.

Referring again to FIGS. 3A and 3B, the energy source 210 or the optical system 212 can include any suitable filter able to select, or at least partially select, certain wavelengths or wavelength bands from energy source 210. In certain types of filters, the filter may block a specific set of wavelengths. It is also possible that undesired wavelengths in the energy from energy source 210 may be wavelength shifted in ways known in the art so as to enhance the energy available in the desired wavelength bands. Thus, filter(s) may include elements designed to absorb, reflect or alter certain wavelengths of electromagnetic radiation. For example, filter(s) may be used to remove certain types of wavelengths that are absorbed by surrounding tissues.

Generally, optical system 212 of FIGS. 3A and 3B functions to receive radiation from the source 210 and to focus/concentrate such radiation to one or more beams 220 directed to a selected one or more treatment or target portions 214 of volume V, the focus being both to the depth d and spatially in the area A (see FIG. 3A). Some embodiments use such an optical system 212, and other embodiments do not use an optical system 212. In some embodiments, the optical system 212 creates one or more beams which are not focused or divergent. In embodiments with multiple sources, optical system 212 may focus and/or concentrate the energy from each source into one or more beams and each such beam may include only the energy from one source or a combination of energy from multiple sources.

If an optical system 212 is used, the energy of the applied light can be concentrated to deliver more energy to target portions 214. Depending on system parameters, portions 214 may have various shapes and depths as described above.

The optical system 212 as shown in FIGS. 3A and 3B may focus energy on portions 214 or on a selected subset of portions 214 simultaneously. Alternatively, the optical system 212 may contain an optical or mechanical-optical scanner for moving radiation focused to depth d to successive portions 214. In another alternative embodiment, the optical system 212 may generate an output focused to depth d and may be physically moved on the skin surface over volume V, either manually or by a suitable two-dimensional or three-dimensional (including depth) positioning mechanism, to direct radiation to desired successive portions 214. For the two later embodiments, the movement may be directly from one portion to be focused on to the next portion to be focused on or the movement may be in a standard predetermined pattern, for example a grid, spiral or other pattern, with the EMR source being fired only when over a desired portion 214.

Where an RF or other non-optical EMR source such as acoustic is used as energy source 210, the optical system 212 can be a suitable system for concentrating, or focusing such energy, for example a phased array, and the term “optical system” should be interpreted, where appropriate, to include such a system. See Tables 3A,B and 4A,B above for exemplary ranges of parameters for ultrasound and radiofrequency based systems, respectively.

It is generally necessary in a scattering medium such as skin to focus, in order to achieve a focus at a deeper depth d. The reason for this is that scattering prevents a tight focus from being achieved and results in the minimum spot size, and thus maximum energy concentration, for the focused beam being at a depth substantially above that at which the beam is focused. The focus depth can be selected to achieve a minimum spot size at the desired depth d based on the known characteristics of the skin.

As set forth above, the system 208 can also include a cooling element 215 to cool the surface of the skin 200 over treatment volume V. As shown in FIGS. 3A and 3B, a cooling element 215 can act on the optical system 212 to cool the portion of this system in contact with the patient's skin, and thus the portion of the patient's skin in contact with such element. In some embodiments intended for use treating superficial sources of pain in tissue, the cooling element 215 might not be used or, alternatively, might not be cooled during treatment (e.g., cooling only applied before and/or after treatment). In some embodiments, cooling can be applied on a portion of the skin surface, for example, between treatment islets or where the treatment islets are to be formed. In some embodiments, cooling of the skin is not required and a cooling element might not be present on the hand piece. In other embodiments, cooling may be applied only to the portions of tissue between the treatment islets in order to increase contrast.

The cooling element 215 can include a system for cooling the radiation system (and hence the portion in contact with the skin tissue) as well as a contact plate that touches the patient's skin tissue when in use. The contact plate can be, for example, a flat plate, a series of conducting pipes, a sheathing blanket, or a series of channels for the passage of air, water, oil or other fluids or gases. Mixtures of these substances may also be used, such as a mixture of water and methanol. For example, in one embodiment, the cooling system can be a water-cooled contact plate. In another embodiment, the cooling mechanism may be a series of channels carrying a coolant fluid or a refrigerant fluid (for example, a cryogen), which channels are in contact with the patient's skin 200 or with a plate of the apparatus 208 that is in contact with the patient's skin. In yet another embodiment, the cooling system may comprise water spray or refrigerant fluid (for example R134A) spray, a cool air spray or air flow across the surface of the patient's skin 200. In other embodiments, cooling may be accomplished through chemical reactions (for example, endothermic reactions), or through electronic cooling, such as Peltier cooling. In yet other embodiments, cooling mechanism 215 may have more than one type of coolant, or cooling mechanism 215 and/or contact plate may be absent, for example, in embodiments where the tissue is cooled passively or directly, for example, through a cryogenic or other suitable spray. Sensors or other monitoring devices may also be embedded in cooling mechanism 215 or other portions of the hand held device, for example, to monitor the temperature, or to determine the degree of cooling required by the patient's skin 200, and the cooling mechanism 215 or the hand held device may be manually or electronically controlled.

In certain cases, cooling mechanism 215 may be used to maintain the surface temperature of the patient's skin 200 at its normal temperature, which may be, for example, about 37° C. or about 32° C., depending on the type of tissue being heated. In other embodiments, cooling mechanism 215 may be used to decrease the temperature of the surface of the patient's skin 200 to a temperature below the normal temperature of that type of tissue. For example, cooling mechanism 215 may be able to decrease the surface temperature of tissue to, for example, a range between 25° C. and −5° C. In other embodiments, a plate can function as a heating plate in order to heat the patient's skin. Some embodiments can include a plate that can be used for cooling and heating.

A contact plate of the cooling element 215 may be made out of a suitable heat transfer material, and also, where the plate contacts the patient's skin 200, of a material having a good optical match with the tissue. Sapphire is an example of a suitable material for the contact plate. Where the contact plate has a high degree of thermal conductivity, it may allow cooling of the surface of the tissue by cooling mechanism 215. In other embodiments, contact plate may be an integral part of cooling mechanism 215, or may be absent. In some embodiments, such as shown in FIGS. 3A and 3B, energy from energy source 210 may pass through contact plate. In these configurations, contact plate may be constructed out of materials able to transmit at least a portion of energy, for example, glass, sapphire, or a clear plastic. In addition, the contact plate may be constructed in such a way as to allow only a portion of energy to pass through contact plate, for example, via a series of holes, passages, apertures in a mask, lenses, etc. within the contact plate. In other embodiments, energy may not be directed through a cooling mechanism 215.

In certain embodiments, various components of system 208 may require cooling. For example, in the embodiment shown in FIGS. 3A and 3B, energy source 210, optics 212, and filter may be cooled by a cooling mechanism (not shown). The design of cooling mechanism may be a function of the components used in the construction of the apparatus. The cooling element 215 for the patient's skin 200 and the cooling element for the components of the system 208 may be part of the same system, separate systems or one or both may be absent. Cooling mechanism for the components of the system 208 may be any suitable cooling mechanism known in the art. Cooling of the components may be accomplished through convective or conductive cooling, for example. In some embodiments, the cooling element can prevent optics 212 from overheating due absorption of radiation.

Typically cooler 215 is activated before source 210 to pre-cool the patient's skin to a selected temperature below normal skin temperature, for example −5° C. to 10° C., to a depth of at least the DE junction 206, and preferably to depth d to protect the entire skin region 220 above volume V (see, e.g., FIG. 3B). However, if pre-cooling extends for a period sufficient for the patient's skin to be cooled to a depth below the volume V, and in particular if cooling continues after the application of radiation begins, then heating will occur only in the radiated portions 214, each of which portions will be surrounded by cooled skin. Therefore, even if the duration of the applied radiation exceeds the thermal relaxation time for portions 214, heat from these portions will be substantially contained such that thermal damage beyond these portions is avoided. Further, while nerves may be stimulated in portions 214, the cooling of these nerves outside of portions 214 will, in addition to permitting tight control of damage volume, also block pain signals from being transmitted to the brain, thus permitting treatments to be effected with greater patient comfort, and in particular permitting radiation doses to be applied to effect a desired treatment which might not otherwise be possible because of the resulting pain experienced by the patient.

A number of different devices and structures can be used to spatially modulate and/or concentrate radiation in order to generate islets of damage (e.g., damaged portions of the volume separated from other damaged portions of the volume by non-treated portions of the volume) for treating inflammation and/or pain in the tissue. For example, the devices can use reflection, refraction, interference, diffraction, and deflection of incident light to create treatment islets. Such devices are described in greater detail in the applications listed herein that are incorporated by reference in their entirety.

In other embodiments, spatially separated islets of treatment (e.g., islets of damage) can be created by applying to the skin surface a desired pattern of a topical composition containing a preferentially absorbing exogenous chromophore. The chromophore can also be introduced into the tissue with a needle, for example, a micro needle as used for acupuncture. In this case, the radiation energy may illuminate the entire skin surface where such pattern of topical composition has been applied. Upon application of appropriate radiation, the chromophores can heat up, thus creating islets of treatment (e.g., islets of damage) in the skin. Alternatively, the radiation energy may be focused on the pattern of topical composition. A variety of substances can be used as chromophores including, but not limited to, carbon, metals (Au, Ag, Fe, etc.), organic dyes (Methylene Blue, Toluidine Blue, etc.), non-organic pigments, nanoparticles (such as fullerenes), nanoparticles with a shell, and carbon fibers, etc. The desired pattern can be random and need not be regular or pre-determined. The treatment pattern can vary as a function of the desired treatment area and can be generated ad hoc.

The lattices can be produced using non-optical sources. For example, ultrasound, microwave, radio frequency and low frequency or DC EMR sources can be used as energy sources to create lattices of radiation treated islets. Also, various optical and/or non-optical sources can be combined, such as visible light, acoustic energy, ultrasound, and shockwaves (e.g., formed by the application or heat, acoustic energy, ultrasound or other forms of energy). In addition, the sources can be combined with various mechanical stimuli, such as a vacuum or vibrating mechanism, to improve and facilitate the treatment of tissue.

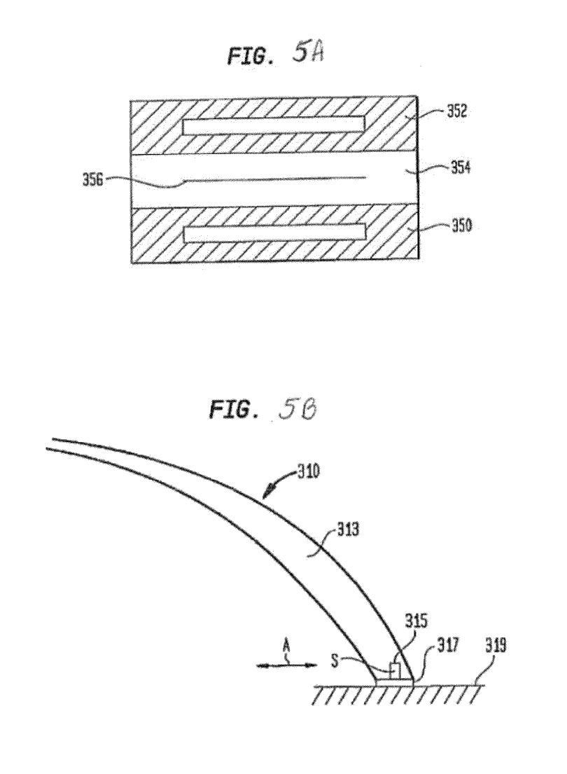

A number of different devices and structures can be used to generate islets of treatment in the skin. FIG. 4 illustrates one system for producing the islets of treatment on the skin 280. An applicator 282 is provided with a handle so that its head 284 can be near or in contact with the skin 280 and scanned in a direction 286 over the skin 280. The applicator 282 can include an islet pattern generator 288 that produces a pattern of damaged islets (e.g., damaged portions separated from non-treated portions or less treated portions created by treatment with a radiation source such as EMR). The islet pattern generator 288 can provide areas of enhanced permeability in the stratum corneum (e.g., a pattern of enhanced permeability) or an arrangement 290 of islets 292 on the surface of the skin 280. In other embodiments, the generator 288 can produce thermal islets, damage islets, or photochemical islets into the epidermis and/or in the dermis and/or in the DE junction.

In one embodiment, the applicator 282 includes a motion detector 294 that detects the scanning of the head 284 relative to the surface 296 overlying the target tissue. This generated information is used by the islet pattern generator 288 to ensure that the desired fill factor or islet density and power is produced at the skin surface 296. For example, if the head 284 is scanned more quickly, the pattern generator responds by imprinting islets more quickly.

According to one embodiment, an apparatus can include a light emitting assembly for applying radiation energy (e.g., optical energy) to the target area of the patient's skin, a sensor for determining the speed of movement of the head portion across the target area of the patient's skin, and circuitry in communication with the sensor for controlling the optical energy in order to create islets of treatment. The circuitry can control, for example, pulsing of the optical energy source based on the speed of movement of the head portion across the skin in order to create islets of treatment. In another embodiment, the circuitry can control movement of the energy source within the apparatus based on the speed of movement of the head portion across the skin in order to treat certain areas or portions of the skin, while not exposing other areas or portion of the skin to treatment, thereby create islets of treatment or stated differently damaged portions of the volume of tissue separated from one another by non-treated portions of the volume.

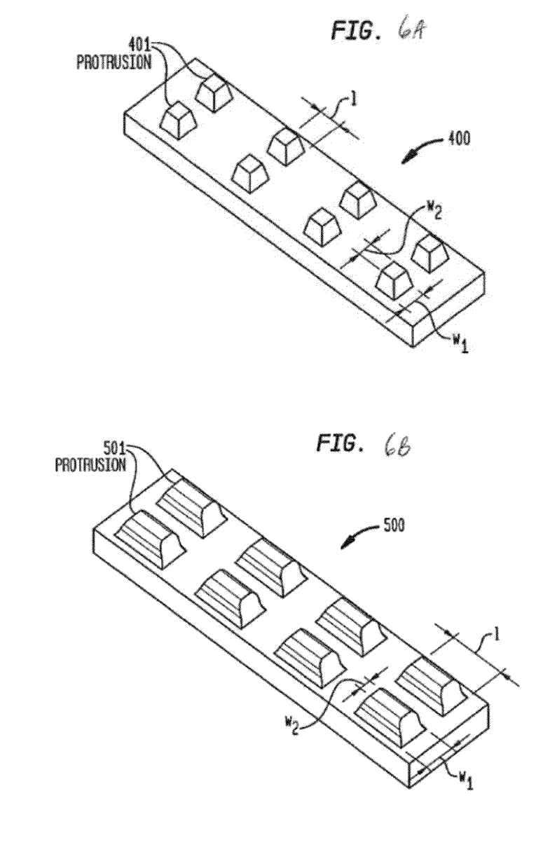

FIG. 5A is a bottom view of an embodiment that includes one or more speed sensor(s) for measuring the speed of movement of the hand piece across the patient's skin. The speed sensor shown in FIG. 5A can be used, for example, in the embodiment of FIG. 5B. That is, the hand piece 310 of FIG. 5B can include a housing 310 and a diode laser bar 315 (or more than one diode laser bars). FIG. 5A shows a bottom view of a hand piece that is equipped with a speed sensor 350, 352.

A number of types of speed sensors can be used to measure the hand piece speed relative to the skin surface. For example, the speed sensor can be an optical mouse, a laser mouse, a wheel/optical encoder, or a capacitive imaging array combined with a flow algorithm similar to the one used in an optical mouse. A capacitive imaging array can be used to measure both hand piece speed and to create an image of the treated area. Capacitive imaging arrays are typically used for thumbprint authentication for security purposes. However, a capacitive imaging array can also be used to measure the hand piece speed across the skin surface. By acquiring capacitive images of the skin surface at a relatively high frame rate (for example, 100-2000 frames per second), a flow algorithm can be used to track the motion of certain features within the image and calculate speed.

In the embodiment of FIG. 5A, two capacitive imaging arrays 350, 352 are located on the bottom of the hand piece, with one on each side of the treatment window 354. The diode laser bar 356 output is directed through the treatment window. The treatment window 352 can include, for example, a cooling plate or the like. The orientation of the capacitive imaging arrays 350, 352 can vary in different embodiments. As the device is moved, both capacitive imaging arrays 350, 352 measures the speed of the hand piece across the patient's skin. The configuration can include circuitry that is in communication with the capacitive imaging arrays 350, 352 to measure the speed and determine an appropriate rate for firing the light source (e.g., diode laser) based on that speed. The circuitry, therefore, can also be in communication with the laser in order to pulse the laser at an appropriate speed. The speed sensor incorporated in the hand piece, therefore, can provide feedback to the laser pulse generator. In some embodiments, after an initial pulse of radiation, the pulsing of the diode laser bar 356 might not be enabled until the capacitive imaging arrays 350, 352 sense movement of the hand piece over the skin. This circuitry can be located in the hand piece in some embodiments or, in other embodiments, in a base unit. When the diode laser bar 356 is enabled for firing by the user (for example by depressing a footswitch or by turning a handheld device on), a laser pulse generator for the laser fires the laser at a rate proportional to the hand piece speed. In some embodiments, such speed sensors work in combination with inflammation detectors to provide the desired amount of treatment in a region of inflammation.

In operation, the speed sensor embodiment described above can be used to create a uniform matrix of treatment islets by manually moving a hand piece that includes a single diode laser bar (or multiple diode laser bars) across the skin surface and pulsing the laser at a rate proportional to the hand piece speed. For example, decreasing the time interval between laser pulses as the hand piece speed increases can be used to keep a consistent matrix of lines of islets of treatment on the skin. Similarly, increasing the time interval between laser pulses as the hand piece speed decreases can be used to keep a consistent matrix of lines of islets of treatment on the skin. The treatment head, including treatment window or light aperture of the hand piece, can be rotated to vary the spacing between islets of treatment in the direction orthogonal to hand piece movement.

In addition to measuring hand piece speed, the capacitive imaging arrays 350, 352 can also image the skin after the line of islets of treatment has been created in order to view the treatment results. Acquired images can be viewed in real time during treatment. The hand piece can include, for example, a display that shows the treatment area of the skin under the cooling plate. Alternatively, the acquired images can be stored in a computer for viewing after the treatment is complete. In some embodiments, the system can be configured to display images from both sensors, so that the hand piece can be moved either forward or backward.

Another embodiment involves the use of imaging optics to image the patient's skin. In one embodiment, the imaging optics measures heat emitted by a region of inflammation at the skin surface and/or underlying the skin surface and use that information to determine application radiation (e.g., application of EMR or the like) in order to optimize performance. For instance, some inflammation and/or pain treatments require that the islet formation rate be accurately measured and its effect be analyzed in real time. The skin surface imaging system can detect the size of treatment volumes created with techniques proposed in this specification for creating treatment islets (e.g., for creating damaged tissue portions separated by non-treated portions and/or less treated portions in a volume of inflamed tissue). For this purpose, a capacitive imaging array can be used (optionally, in combination with an image enhancing lotion and a specially optimized navigation/image processing algorithm) to locate regions of inflammation and/or measure and control the application rate in light of the measured inflammation.

The use of a capacitive imaging array is set forth above in connection with FIG. 5A. Such capacitive image arrays can be used, for example, within the applicator 282 of FIG. 4 according to this embodiment. As set forth above, in addition to measuring hand piece speed, the capacitive imaging arrays 350, 352 (FIG. 5A) can also image the skin. Acquired images can be viewed in real time during treatment via a display window of the device.

One example of a suitable capacitive sensor for this embodiment is a sensor having an array of 8 image-sensing rows by 212 image-sensing columns. Due to inherent limitations of capacitive array technology, a typical capacitive array sensor is capable of processing about 2000 images per second. To allow for processing skin images in real time, an orientation of the sensor can be selected to aid in functionality. In one embodiment, for instance, the images are acquired and processed along the columns. This allows for accurate measurement of velocity up to about 200 mm/s.

For the sensor to function reliably and accurately, the skin surface can optionally be treated with an appropriate lotion. In some embodiments, a properly selected lotion can improve the light-based skin treatment and navigation sensor operation. A lotion may be optically transparent to the selected wavelength, provide image enhancement to a sensor, and/or function as a friction reduction lubricant.

The sensor can also function as a contact sensor. This allows for real time determination of immediate contact of a hand piece with the patient's skin. The combination of hardware and software allows this determination within one image frame. The algorithm measures in real time skin contact and navigation parameters (position, velocity and acceleration) along the x-axis and y-axis. This enhances the safety of light treatment on human skin by allowing for the control of the velocity and the quality of skin contact. The quality of contact can be a function of lotion type and/or pressure applied to the treatment device.

Some embodiments use one or more diode laser bars as an EMR source of radiation. Any suitable diode laser bar can be used including, for example, 10-100 W diode laser bars. Other sources (e.g., LEDs and diode lasers with SHG) can be substituted for the diode laser bar with suitable modifications to the optical and mechanical sub-systems.

FIG. 5B shows an embodiment of the invention using a diode laser bar. Many other embodiments can be used within the scope of this disclosure. In this embodiment, the hand piece 310 includes a housing 313, a diode laser bar 315, and a cooling or heating plate 317. The housing 313 supports the diode laser bar 315 and the cooling or heating plate 317, and the housing 313 can also support control features (not shown), such as a button to fire the diode laser bar 315. The housing 313 can be made from any suitable material, including, for example, plastics. The cooling plate, if used, can remove heat from the patient's skin. The heating plate, if used, can heat the patient's skin. The same plate can be used for heating or cooling, depending on whether a heat source or source of cooling is applied to the plate.

Referring again to FIG. 5B, the plate 317 can be of any type, such as those set forth above, in which light from an EMR source can pass through the plate 317. In one embodiment, the plate 317 can be a thin sapphire plate. In other embodiments, other optical materials with good optical transparency and high thermal conductivity/diffusivity, such as, for example, diamond, can be used for the plate 317. The plate 317 can be used to separate the diode laser bar 315 from the patient's skin 319 during use. In addition, the plate 317 can provide cooling or heating to the patient's skin, if desired. The area in which the plate 317 touches the patient's skin can be referred to as the treatment window. The diode laser bar 315 can be disposed within the housing 313 such that the emitters are in close proximity to the plate 317, and therefore in close proximity to the patient's skin when in use.

In operation, one way to create islets of treatment is to place the housing 313, including the diode laser bar 315, in close proximity to the skin, and then fire the laser. For example, wavelengths near 1200-1700 nm and in the 1390-1430 nm range can be used for creating islets of treatment (e.g., damage). Providing cooling to the patient's skin during treatment can enable the creation of subsurface islets of treatment (e.g., damage) that have minimal effect on the epidermis. FIG. 4 shows one possible arrangement 290 of islets on the surface of the skin 280 from the use of such a diode laser bar, where the diode laser bar 315 is pulsed as it moves over the skin in direction A of FIG. 5B.

In another embodiment, the user can simply place the hand piece in contact with the target skin area and move the hand piece over the skin while the diode laser is continuously fired to create a series of lines of treatment. For example, using a diode laser bar can create lines of treatment that appear on the skin (one line for each emitter).

In another embodiment, an optical fiber can couple to the output of each emitter of the diode laser bar. In such an embodiment, the diode laser bar need not be as close to the skin during use. The optical fibers can, instead, couple the light from the emitters to the plate that will be in close proximity to the skin when in use.

Multiple diode laser bars can be employed to create a matrix of islets of treatment. For example, multiple diode laser bars can be arranged to form a stack of bars. In operation, the hand piece can be brought close to the skin surface such that the cooling plate is in contact with the skin. The user can simply move the hand piece over the skin as the diode lasers are pulsed to create a matrix of islets of treatment in the skin. The emission wavelengths of the stacked bars need not be identical. In some embodiments, it may be advantageous to mix different wavelength bars in the same stack to achieve the desired treatment results. By selecting bars that emit at different wavelengths, the depth of penetration can be varied, and therefore the islets of treatment spot depth can also be varied. Thus, the lines or spots of islets of treatment created by the individual bars can be located at different depths.

During operation, the user of the hand piece can place the treatment window of the hand piece in contact with a first location on the skin, fire the diode lasers in the first location, and then place the hand piece in contact with a second location on the skin and repeat firing.

In addition to the embodiments set forth above in which the diode laser bar(s) is located close to the skin surface to create islets of treatment (e.g., damaged portions of tissue separated from non-treated portions). A variety of optical systems can be used to couple light from the diode laser bar to the skin. For example, imaging optics can be used to re-image the emitters onto the skin surface, which allows space to be incorporated between the diode laser bar (or the stack of bars) and the cooling plate. In another embodiment, a diffractive optic can be located between the diode laser bar and the output window (i.e., the cooling plate) to create an arbitrary matrix of treatment spots. Numerous exemplary types of imaging optics and/or diffractive optics that can also be used in this embodiment are set forth above.

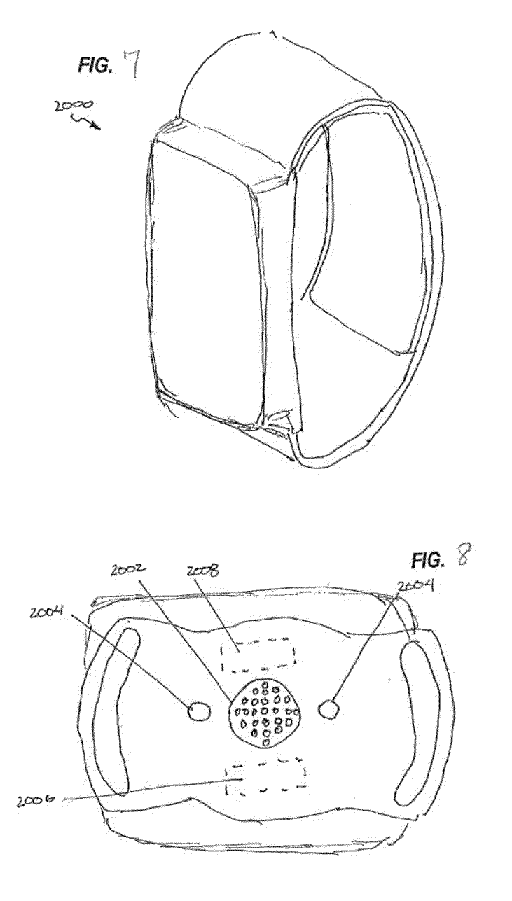

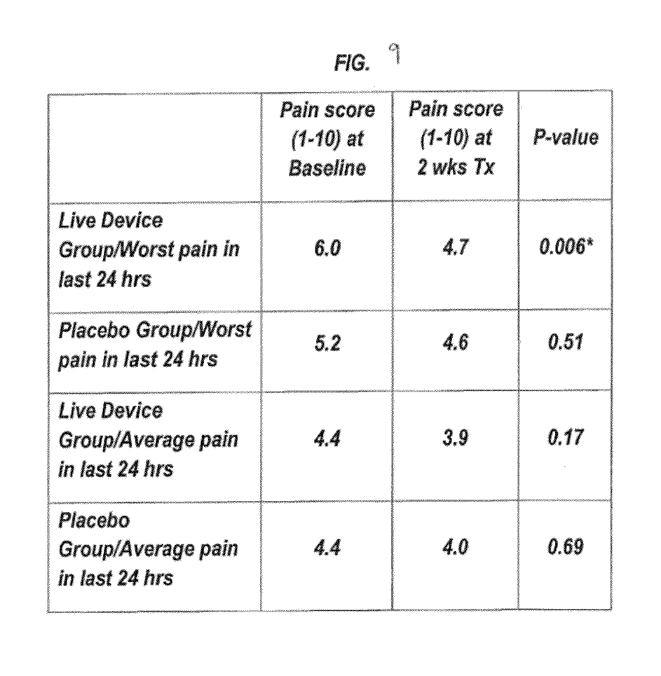

FIG. 7 is a schematic illustration of a wearable device 2000 for pain management in the form of a garment to treat lower back pain. Such a device can be implemented in many wearable forms (e.g., a patch and/or a garment such as a belt, shirt, sock, leg-warmer, pant) that are placed in contact with the desired area to be treated on the patient's body. As shown in the exemplary embodiment, in side view of FIG. 8, the wearable device 2000 includes a source of radiation (e.g., fractional EMR 2002) and can optionally also include sensors 2004, such as an inflammation sensor (e.g., a temperature sensor), to detect the presence or and/or the onset of inflammation. Alternatively, the device may include processor 2006 for implementing an automated feedback mechanism, registering nociceptive activity, and supplying treatment(s) as necessary when the perception of pain is likely felt by the user. Alternatively, or in addition, the device can include an internal timer mechanism 2008, providing timed application of the treatments with desired intensity (energy and density). The interval between applications can range between, for example, about 1 second and about 1 week, about 20 seconds and about 1 day, about 1 min and about 12 hours, about 30 minutes and about 6 hours.

In addition to the described methods and devices for treating inflammation and/or pain by application of radiation to a region including inflamed tissue to create damaged portions of the tissue volume (e.g., damaged portions of the inflamed tissue volume, damaged portions of the tissue adjacent the volume of inflamed tissue, or damaged portions of both the volume of inflamed tissue and the volume of the tissue adjacent the inflamed tissue volume) separated by non-treated portions of tissue, drugs and/or other substances can also be introduced to the body of the subject. For example, in some embodiments, a corticotropin-releasing hormone (CRH) is introduced to the body of the subject by any of a number of methods including, for example, oral delivery, topical application, injection, and/or injections. For example, Adrenocorticotropic hormone (ACTH), or corticotropin, is a hormone available under the trade name ACTHAR that is produced in and released from the pituitary gland. ACTH is normally released from the pituitary in response to stimulation with corticotropin-releasing hormone (CRH), a hormone produced in the hypothalamic region of the brain during various types of stress or pain. The principal action of ACTH is to stimulate the synthesis and release of steroid hormones from the adrenal glands, which lie on the surface of the kidneys. In one embodiment, the CRH hormone is in a topical. The topical may be disposed on the location of the region of tissue containing the inflamed tissue volume prior to, simultaneous with, or after applying radiation. In one embodiment, the CRH hormone is injected into the location of the inflamed tissue volume. In still another embodiment, the CRH hormone is ingested (e.g., orally ingested) prior to treating pain and/or inflammation by fractionally applying radiation to a volume of inflamed tissue.

Micro-holes (e.g., ablated fractional portions) can be used to facilitate the delivery of drugs or other substances (e.g., CRH hormones) through the skin or other soft tissues. For example, in one embodiment, a mixture containing an analgesic drug (or drugs) and/or other substances having low absorption rates can be applied to the surface of the skin in an area that has been treated with radiation energy to create and array of micro-holes (e.g., portions of ablated tissue of the volume separated by a non-treated portion of the volume). Treatments according to this embodiment may involve treatments of one or more different anatomical sites of the human body, such as knee joints, hips, shoulders, etc. and multiple target sites or tissue types can be treated simultaneously.

Presently, many potential therapies for the treatment of pain are undesirable due to the toxic effect of drugs taken orally, by injection or intravenously. Similarly, many approved painkillers are also taken orally, by injection, intravenously, or superficially (e.g., via topical application) on a regular basis (e.g., daily or even hourly) for the treatment of skin or other superficial organ pain. Applying the treatment substance having a low dissolving rate inside a human body has been successfully used for the treatment of long lasting pain or for preventative purposes. In most such cases, the substance is a matrix of tablets which dissolve slowly and release embedded medicine to maintain the necessary concentration locally.

Pain treatment substances having a low dissolving rate can be applied to micro-holes, such as micro channels, for the treatment of human skin and other diseases. The uptake of the treatment substance can be enhanced by embedding the substance within the micro-holes using chemical enhancers (e.g., polar solvents such as decylmethylsulfoxide and polyenic antibiotics to enhance membrane permeability). The uptake of the treatment substance can also be aided by use of mechanical energy or other energy, for example, positive and/or negative pressure, magnetic fields applied to magnetic substances, electric fields applied to electrically charged substances (e.g., iontophoresis), local skin heating, massage or other mechanical manipulation of the tissue, sprays (e.g., high pressure sprays with small droplets), light waves or other EMR-induced stress, acoustic waves including sonophoresis, and other forms of ultrasound. The treatment method may involve (but would not necessarily be limited to) one or more steps of treatment with single wavelengths, and may also be applied in the course of two or more repetitions of the treatment procedure in one or more treatment sessions. Multiple wavelengths may also be used, depending on the application, which may be applied using the same or different light sources.

Many substances can be used, including, for example, pure substances, mixtures containing one or more active compounds; and compounds in an active or inactive matrix. The substance applied can be in various forms, including, without limitation, liquid, solid, gel or aerosol forms.

Drugs or other substances having high absorption rates can also be applied, but the mechanism is presently thought to work more beneficially with drugs having a low absorption rate. Furthermore, in other embodiments, a substance normally having a high dissolving rate can be applied slowly, because the dissolving rate can be dictated by the active ingredients and/or inactive ingredients. Thus, a mixture having a low dissolving rate can be manufactured to include an ingredient that normally has a high dissolving rate.

In some embodiments, the treatments involve three steps. First, micro-holes (e.g., ablated portions of tissue separated by non-treated tissue) are created in the tissue, such as human skin. The micro-holes are created at the selected anatomical location. Second, the substance is embedded in the micro-holes. This step can be performed by various methods, including, without limitation, simple diffusion, vesicle/particle transporters, physical mechanisms, chemicals, or electrical mechanisms, electroporation, iontoporation, sonophoresis, magnetophoresis, photomechanical waves, niosomes, and transfersomes. Third, the substance is sealed within the micro hole. This can be accomplished by various methods, including, without limitation, natural healing, healing creams, covering with, e.g., tapes or strips, and sutures. The process may need to be repeated several times depending on the application.

Generally, the depth of the micro-holes increases proportionally to an increase of energy per beam. Suitable micro-holes may be traversed from the epidermis and through the hypodermis, for example. Prior experiments have demonstrated, among other things, that the micro-holes (e.g., ablated fractions) can be used for incorporation of drugs and/or other substances, into skin or other tissue in vivo. For example, a drug or other substance having a low absorption rate can be placed in a set of micro-holes for incorporation into the body over a period of time, such as one or more months.

Tissue can be treated by, for example, cooling tissue (e.g., muscle tissue, fat tissue, dermal tissue, and/or epidermal tissue), which can include cooling to a temperature below normal body temperature, and preferably below the phase transition temperature of at least some fraction of the lipid content of fatty cells and below the phase transition temperature of at least some fraction of the water content in dermal and/or epidermal cells. The phase transition temperature of at least some of the fraction of the lipid content of fatty cells is substantially higher than the freezing temperature of water-containing tissue. Optionally, cooling of the muscle tissue, the fat tissue, the dermal tissue, and/or the epidermal tissue can be preceded by or followed by heating the tissue (e.g., the muscle tissue, the fat tissue, the dermal tissue, or the epidermal tissue) to a temperature below its damage threshold

The cooling temperature gradient can be provided to the local region by exposing the subject's skin to a cooling element. The cooling energy can travel to the target tissue depth (e.g., the lipid-rich tissue) via the subject's tissue. The lipid-rich adipocytes may be contained in, for example, subcutaneous adipose tissue (adipocyte), sebaceous glands and sebocytes. Selective disruption of adipocytes can result from localized crystallization of highly saturated fatty acids upon cooling at temperatures that do not induce crystallization of water in other cells. In some embodiments, the cooling element temperature can provide controlled damage to the lipid-rich adipose tissue. Crystallization of lipid in adipocytes can occur at a temperature below about 37° C., for example from about 20° C. to about 0° C., or from about 10° C. to about 20° C. Likewise, epidermal and/or dermal tissue can be damaged through crystallization of water in the epidermal tissue and/or dermal tissue, which can occur at or below 0° C., for example.

Treatment of tissue can target, for example, joint tissue, muscle tissue, fat tissue, dermal tissue, or epidermal tissue in a volume of inflamed tissue. The target depth can include at least one of the reticular dermis, subcutaneous fat and muscle. The reticular dermis may be located at about 0.25 mm below the surface of the skin, or at a deeper depth below the surface of the subject's skin. For example, the reticular dermis can have a depth that ranges from about 1 mm to about 3 mm in depth. Subcutaneous fat can also be called or referred to as the hypodermis.

Generally, the time for changing the temperature at the skin surface (e.g., cooling) must be long enough to allow the temperature gradient to flow to the epidermis, from the epidermis to the dermis, and/or from the epidermis to the dermis to the subcutaneous adipose layers in order to achieve the desired temperature at the layer that provides the desired treatment.

When the targeted tissue is subcutaneous adipose, it is cooled to a temperature below the temperature for lipid crystallization (e.g., below 37° C., for example from about 20° C. to about 0° C.). When the targeted tissue is epidermal tissue or dermal tissue it is cooled to a temperature below the temperature for crystallization of water, at or below 0° C., for example. In some embodiments, the skin surface cooling temperature and cooling time can be adjusted to control depth of treatment, for example the anatomical depth to which tissue (e.g., epidermal, dermal, or adipose tissue) is affected. Heat diffusion is a passive process, and the body core temperature is nearly always close to 37° C. Therefore, generally, for at least part of the time during which cooling is performed the skin surface temperature must be lower than the desired target temperature for treatment in the target region.

In order to practice the treatment of tissue, a thermal element is employed to treat the target volume (e.g., the inflamed volume). The thermal element can be solely for cooling or can cycle cooling and heating. The thermal element can be employed as a thermal control element. The thermal element can be a cooling element such as a contact tip or a contact agent that is applied in contact with or proximal to the subject's skin in a target region. Contact with the cooling element can create a temperature gradient within the target volume sufficient to selectively damage tissue and/or disrupt adipocytes and/or disrupt sebaceous glands therein. Application of the cooling element to the subject's skin may be repeated a plurality of times until the desired damage is achieved. Where the cooling element is a contact tip it may be coupled to or contain a cooling agent. Cooling elements of the present invention can contain cooling agents in the form of a solid, liquid or gas. Solid cooling agents can include, for example, thermal conductive materials, such as metals and/or metal plates and solid cooling agents can also include glass, gels and ice or ice slurries. Liquid cooling agents can comprise, for example, saline, glycerol, alcohol, or water/alcohol mixtures, for example. Where the cooling element includes a circulating cooling agent, preferably the temperature of the cooling agent is constant. Salts can be combined with liquid mixtures to obtain desired temperatures. Gasses can include, for example, cold air or liquid nitrogen. In one embodiment, the cooling element is applied such that direct contact is made with a subject, via either the agent or the element. In another embodiment, direct contact is made via the agent alone. In yet another embodiment, no direct contact is made via either the agent or the element and cooling is a carried out by proximal positioning of the cooling element and/or agent. Preferably, the temperature of the cooling agent is less than about 20° C.

The cooling agent can be applied in a pulsed or continuous manner. The cooling element and/or agent can be applied by all conventional methods known in the art, including topical application by spray of a cooling agent in liquid form, gas or particulate solid material. The cooling may be applied externally.

Where the thermal element cycles cooling and heating, the cooling unit can be a thermoelectric element, an enclosure with cooling agent, a stream of cold gas (or liquid) or other cooling unit known in the art. Phase-changing materials can also be used for cooling. Skin surface temperature during the cooling phase should be maintained within the range of from about −5° C. to about 30° C. or from about 0° C. to about 20° C. Tissue temperature in the heating phase should be maintained in the range of from about 25° C. to about 55° C., or from about 25° C. to about 40° C., or from about 35° C. to about 45° C. In one embodiment of the disclosure, optical radiation is used in the heating phase of the cycle. In another embodiment, electromagnetic radiation (EMR) is used on the heating phase of the cycle. In this embodiment, the energy source can be a laser, an LED, a lamp (discharge, halogen or other), or a combination or an array thereof. For example, the optical radiation can travel through or around the thermoelectric element. The spectral composition of the source can be either narrow- or broad-band, with the range of wavelengths between 400 nm and 2000 nm. Spectral filtration can be used for further modifying spectral composition of the beam in order to achieve optimal penetration. The wavelengths used for a particular application will depend on the target tissue, the depth of the tissue and other factors. In one embodiment, the light source is operated in the continuous wave (CW) mode, with a preferred irradiance at the skin surface in the range between 0.1 and 100 W/cm². The thermal cycle is organized in such a way as to maximize efficacy of treatment. Typically, duration of the cooling phase can be between about 10 seconds and about 30 minutes, whereas duration of the heating phase can be between about 1 second and about 4 minutes.

Without being bound to any single theory it is believed that use of a fractional treatment strategy for cooling and/or for cycling cooling and heating sub-volumes of tissue to cause damage in a portion of the tissue volume within a larger target volume of tissue being treated can likewise be effective for treatment of inflammation, pain, and/or pain associated with inflammation. Fractional treatment via cooling and/or via cycled cooling and heating can also be employed to produce controlled damage to the epidermis, dermis, and/or hypodermal layers of the subject's tissue, for example. The controlled damage can promote collagen formation when the sub-volumes of treated tissue heal. Alternatively or in addition, fractional treatment via cooling and/or via cycled cooling and heating may also be employed at a depth or to a depth to treat subcutaneous fatty tissue and/or muscle tissue. Treatment at a depth (e.g., treatment of subcutaneous fatty tissue and/or muscle tissue) and treatments closer to the surface (e.g., treatment of epidermis, dermis, hypodermis) can occur simultaneously, e.g., during a single cooling treatment, or separately.

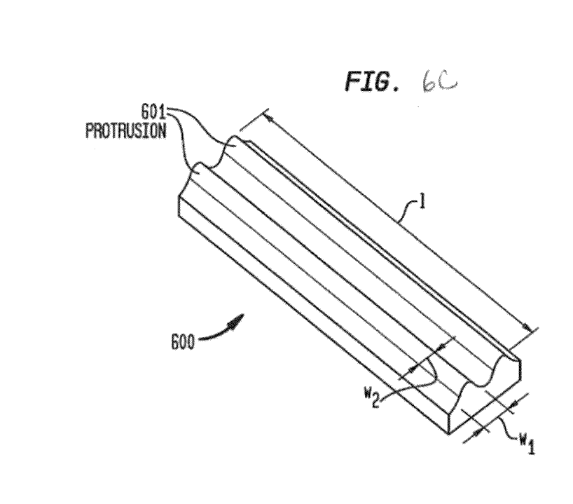

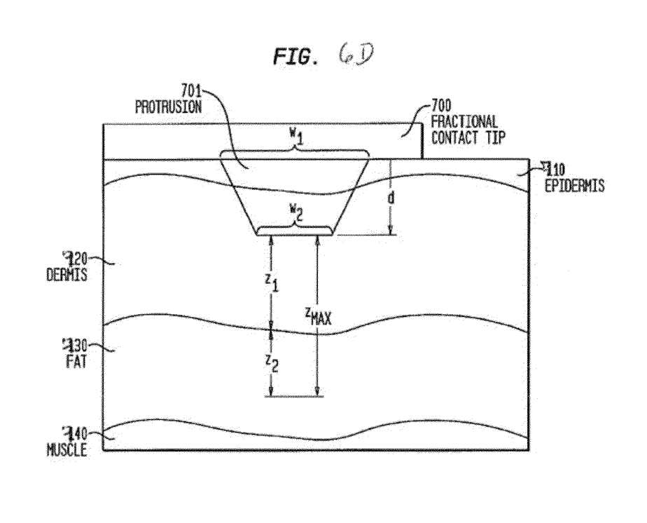

The cooling and/or heating can be applied to the patient's skin fractionally via a contact tip. The contact tip can have any of a number of fractional configurations. Each of the multiple sub-regions can have the same shape or a single contact tip can have a variety of sub-region shapes. The multiple sub-regions for fractional treatment can be positioned in any of a number of patterns (e.g., circular or rectangular). Suitable sub-region protrusion shapes include, for example, squares 401 (FIG. 6A), rectangles 501 (FIG. 6B), and grooves 601 (FIG. 6C). The sub-region shapes and patterns may be selected to suit the region of the body to be treated and/or the quantity of tissue to be treated, for example. One or more of the exemplary contact tips 400, 500, or 600 shown in FIGS. 6A-6C can be adapted to provide multiple sub-regions for cooling alone, multiple sub-regions combining cooling and heating with the some sub-regions for cooling and other sub-regions for heating, and/or multiple sub-regions for cycling cooling with optical energy heating. The one or more sub-regions can be in the form of protrusions that extend to a depth.

FIG. 6D shows a cross section of sub-region of a protrusion of a contact tip similar to the contact tips shown in FIG. 6A-6C or tips with other shape for example cylindrical tips being pressed into a subject's skin such that it presses the subject's epidermis 710 and into the subject's dermis 720 to the depth (d). In one embodiment, where cooling and heating is cycled, one or more of the sub-regions (e.g., cross section of sub-region shown in FIG. 6D) may have one or more integrated optical waveguides or windows that enable dual use of a sub-region for fractional cooling and for fractional light based treatment. It is possible that a single sub-region enables one or more light based transmission there through.

Referring still to FIG. 6D, each of the sub-regions of the fractional contact tip 700 has a depth (d) in addition to its length (l) which is a cross section perpendicular to the length (l) and a first width (w₁) and a second width (w₂) shown, e.g., in FIGS. 6A-6C. The first width (w₁) can vary in the range from about 0.25 mm to about 50 mm. The first width w₁ is related to the depth (zₘₐₓ) of cooling or heating, which can be in the range of from about 0.5 mm to about 25 mm and w₁ is from about 0.5*(zₘₐₓ) to about 3*(zₘₐₓ). The second width (w₂) is smaller than w₁ and can to be in the range of from about 0.5*(zₘₐₓ) to about 2*(zₘₐₓ).

The treatment area (1*w₁) created by each protrusion 701 of the fractional contact tip 700 ranges from about 100μ to about 5 mm, or from about 1 μm to about 2 mm. For example, the fractional contact tip 600 and 700 shown in FIGS. 6C and 6D has at least one sub-region having a depth (d). The depth of treatment created by one or more of the sub-regions of the fractional contact tip (having depth d) ranges from about 200 μm to about 10 mm, or from about 0.5 mm to about 2 mm. The depth of the fractional contact tip may be selected to compress the tissue area being treated. For example, referring to FIG. 6D, the depth and/or exerted pressure may be selected to enable compression of epidermis 710 tissue, dermis 720 tissue and into the subcutaneous fat 730 tissue. In another embodiment, the contact tip fractional depth and/or exerted pressure is selected to enable compression of only the epidermis 710 tissue and the dermis 720 tissue. The sub-regions of the fractional contact tip 700 may be referred to as one or more protrusions 701 that have a depth (d) that measures from about 200 μm to about 10 mm, or from about 0.5 mm to about 2 mm in depth. In one embodiment, the fractional compression is referred to as micro compression.

When a small area of tissue is deformed by compressing or applying pressure to the area or sub-volume of tissue when a thermal and/or photothermal treatment is applied, the penetration of light, heat or cool energy into the tissue is greater than the penetration of the same energy into tissue that is not so deformed. This phenomenon can be used, in particular, to improve fractional thermal and/or photo thermal treatment of tissue and to develop new such treatments. However, the principle is also applicable to non-fractional treatments, where the deformation of a number of small areas of tissue can be used to improve the penetration of energy in non-fractional applications that treat a relatively larger area relative to the size of the deformed areas.

Use of a pressure or compression strategy for cooling, combining cooling and heating, and/or for cycling cooling and heating of areas or sub-volumes of tissue being treated at a depth can limit, reduce, and or eliminate the treatment related pain/discomfort sensed by the body being treated relative to in a non-compression treatment. Because the body senses the applied pressure in addition to the cooling and/or the cycled cooling and heating, application of pressure and/or compression can limit, reduce and/or eliminate the sensation of treatment pain or discomfort sensed by the body being treated relative to a treatment conducted in the absence of fractional pressure and/or compression. Fractional compression reduces or blocks the treatment pain sensation and allows delivery of more cold, heat or light energy without pain. Where pressure and/or compression are applied in a fractional manner, neighboring healthy tissue enables the treated tissue to recover more readily via the multiple non-treated, healthy tissue regions or “sides” that surround the treated tissue.

The depth of the fractional contact tip may be selected to enable treatment of a target depth of at least the depth of the reticular dermis. The reticular dermis can be at about 0.25 mm below the surface of the subject's skin, or it can be deeper. For example, the reticular dermis can be at a depth below the subject's skin that ranges from about 1 mm to about 3 mm, for example.

For the practical application of any treatment it is critical that the treatment time is optimized to reduce the time required for treatment, because a reduced treatment time enables the practitioner to treat each subject quickly and be able to maximize the number of subjects treated in a single day. The cooling time is limited by the time it takes for the drop in temperature gradient to flow through the tissue to the target region (e.g., epidermis, dermis or fatty tissue). The delta in cooling time is a function of the properties of the treatment device (e.g., the tip material such as a sapphire cooling agent), the temperature of the surface of the treatment device contacting with tissue, and the thermal properties of the tissue (e.g., epidermis, dermis, fat, and/or muscle) being treated, thermal diffusion, density, and specific heat capacity and the distance between the skin surface and the treatment area. Micro compression of the skin displaces water from dermal tissue and shortens the distance between the skin surface and the treatment area. The distance between the skin surface and the treatment area can be shortened by up to two times resulting is a faster extraction of heat from the compressed area and thereby decreasing the treatment time. The temperature and time of exposure of the treatment device to the tissue impacts the delta in cooling time. The treatment device can be a contact cooling device made from any of a number materials including sapphire, copper, or aluminum. In an embodiment where cycling of cooling and heating is desired, in a fractional contact tip, one or more sub-regions can include aluminum with integrated optical waveguide or window, for example, where it is desirable combine fractional cooling with fractional light based treatment.

Constraints on cooling treatment include that there is a limit to the amount of cold that the epidermis will tolerate before the epidermis is damaged and/or before the subject experiences discomfort from the treatment. However, where the cooling is introduced to the epidermis according to a fractional technique, non-treated or undamaged tissue surrounds any sub-volume of epidermis and/or dermis tissue damaged by the treatment by exposure to cold during the cooling treatment. The undamaged tissue aids in the healing of any fractionally damaged sub-volume of tissue. The fractional treatment methodology lessens the likelihood of causing permanent damage to the tissue. As a result, during fractional treatment, the sub-regions of skin (e.g., the epidermis and the dermis) can be exposed to a lower temperature and/or to a lower temperature (e.g., about −10° C. to about 0° C.) for a longer period of time without risk of permanent damage to the skin as compared to the same treatment conducted in a non-fractional manner. When a fractional cooling treatment is employed the sub-region of tissue may be treated more severely, because it is on such a small scale (e.g., a micro scale) and is surrounded by healthy tissue to help the damaged tissue to recover. During a fractional cooling treatment the subject can tolerate skin exposure to a lower treatment temperature and/or a lower treatment temperature for a longer period of time as compared to the same treatment conducted in a non-fractional manner. As a result the cool temperature can reach the targeted tissue region (e.g., the dermis and/or the adipocytes in the fat layer) more rapidly fractionally as compared to a treatment conducted in a non-fractional manner.

In some embodiments, a device applies thermal elements for cooling, heating, cycled cooling and heating, and/or a combination of cooling and heating applied to the external surface of a subject's body upon the application of pressure and/or compression to the device. The device can be employed for a long period of time to one or more areas of a subject's body. Suitable devices that apply pressure and/or compression can be, for example furniture, garments, or other appliances that may be located in a region adjacent the subject's skin. In some embodiments, where, for example, the device is a piece of furniture such as a chair, a sofa, and/or a mattress the weight of the subject provide compression to the subject's skin. In other embodiments, the device is a garment and the elasticity of the garment provides compression and/or pressure to the subject's skin. The garment can exert adjustable pressure such as is possible with, for example, an adjustable garment akin to a blood pressure cuff. A garment can be made of a material that provides at least some elasticity, such as, for example, a patch, an adjustable cuff, pants, shirts, dresses, undergarments made of a material having elasticity such as a lycra material. Suitable wearable garments can be made from a relatively inflexible material such as, for example, wood, plastic, metal and paperboard. In other embodiments, all or a portion of the device is inflated to provide pressure to the subject's skin. In other embodiments, all or a portion of the device is under vacuum, which applies pressure and/or compression to the subject's skin. Details regarding thermal elements for cooling, heating, cycled cooling and heating, and/or a combination of cooling and heating that may be employed in accordance with this disclosure may be found at United States Publication No. 2010/0036295 entitled “Method and Apparatus for Fractional Deformation and Treatment of Cutaneous and Subcutaneous Tissue,” which is incorporated by reference herein.

### EXPERIMENTAL RESULTS

A placebo controlled study involving 18 human subjects was conducted. Twelve subjects with 25 anatomical sites of pain were asked to evaluate the effectiveness of a non-ablative fractional skin treatment system for treating pain. The 18 subjects were given the PaloVia® Skin Renewing Laser®, which features a Wavelength of 1410 (+/−20) nm, a Beam Divergence of 0.15 (+/−0.3) mrad, a Pulse Duration of 10 ms, and a Maximum Output Energy of 15 mJ. The expected penetration depth of the PaloVia® Skin Renewing Laser® can range from about 200 microns to about 250 microns. Six additional subjects with 13 anatomical sites of pain were given a look-alike, but non-functional device. All subjects were initially treated and observed in a clinic and were trained on self-treatment.

All subjects were 18 years or older and they included all Fitzpatrick Skin Types (Type I-VI). Each subject had ongoing chronic joint pain that had existed for at least 1 month. For the duration of the study, in the treatment area, subjects had to be willing to abstain from any medical procedure, such as surgery, TENS, ultrasound or radiofrequency treatments, or injections. The subjects also agreed to avoid direct sun exposure to the treatment area(s) for the entire treatment period or to use SPF 30 sunscreen on the treated area(s) for the duration of the study, if going into the sun. The Subjects were also required to refrain from use of pain medication other than OTC NSAIDS for the duration of the treatment period.

Medical histories were taken for all subjects and informed consent was obtained. Exclusion criteria included: previous surgery within 1 year and or injection in the area intended for treatment within the last 6 months; pregnant or nursing; use of prescription pain medications or concurrent drug therapy other than NSAIDs; diseases or skin disorders in treatment area; other recent (2-month) medication changes including anti-inflammatory agents or agents such as glucosamine.

After completing the two-week “treatment” course, placebo group subjects were issued active devices, which they proceeded to use for two additional weeks.

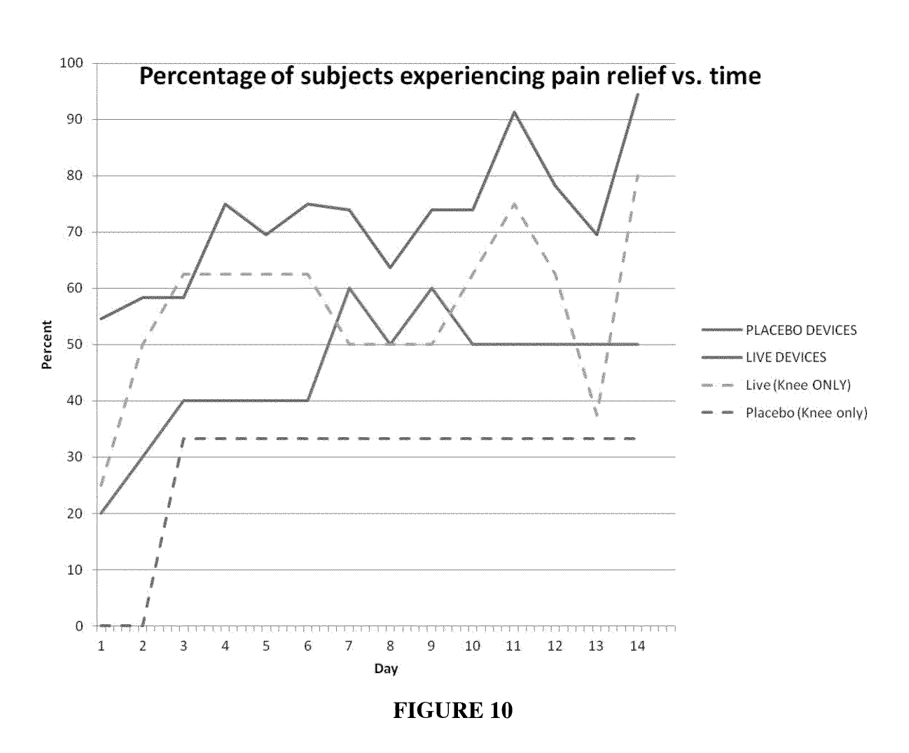

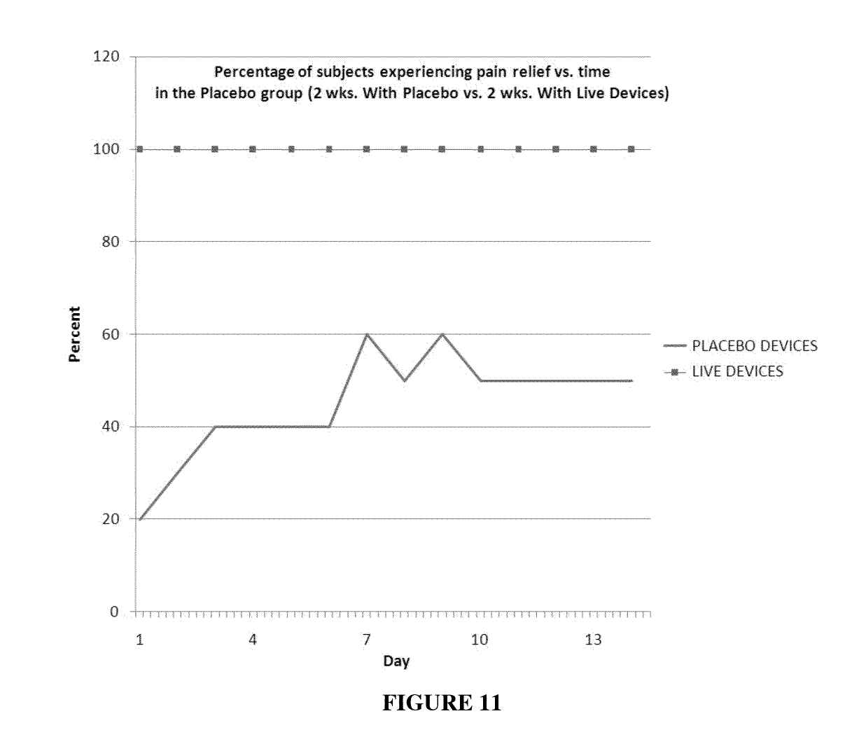

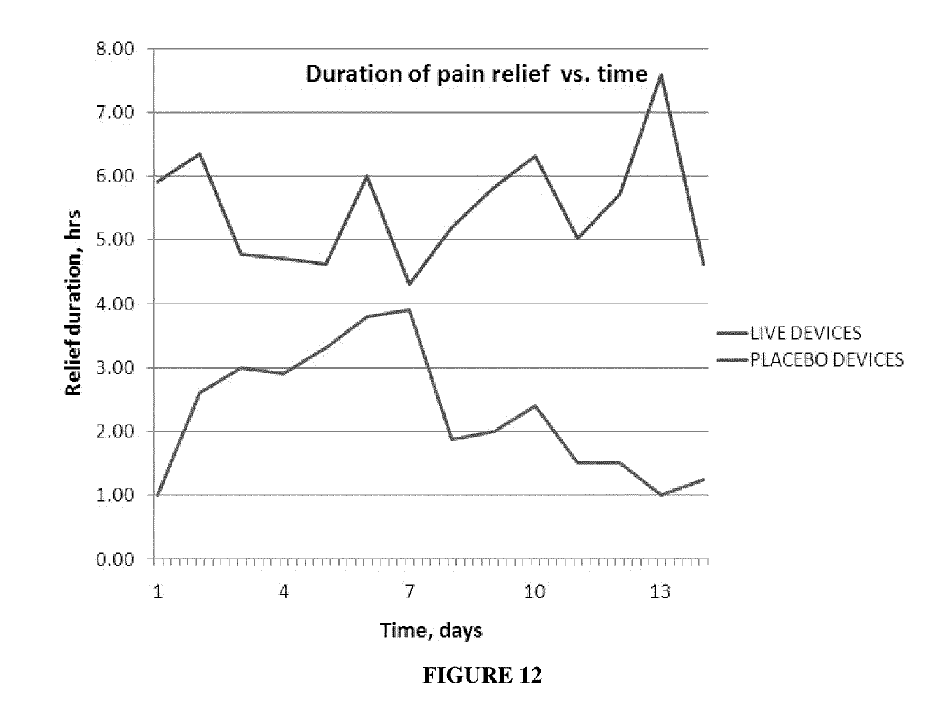

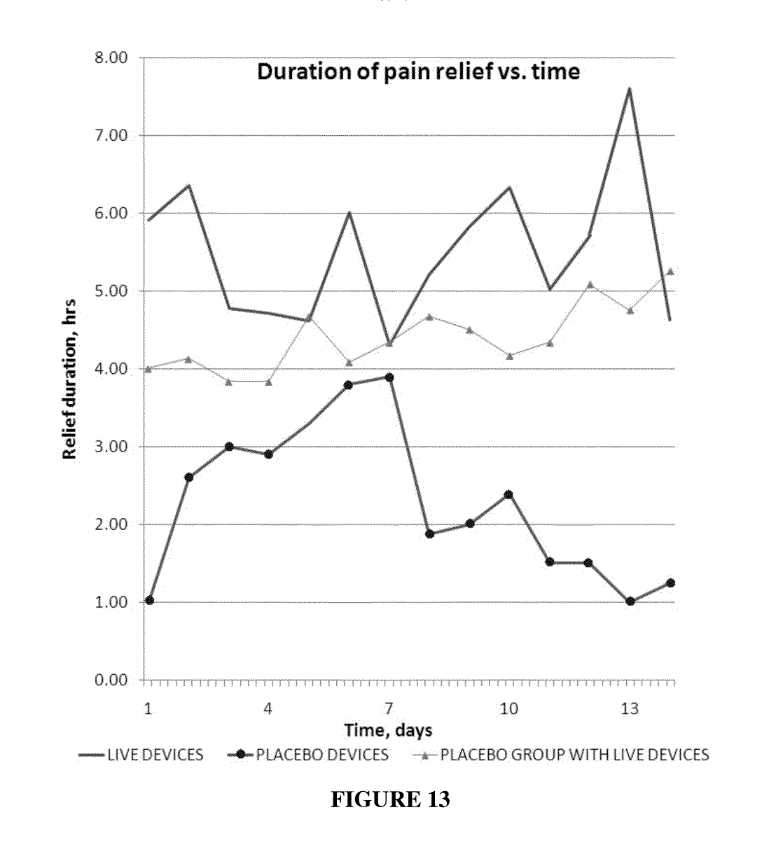

FIG. 9 is a tabulation of results from the study that quantifies relief from “worst pain” and “average pain” reported by the live device group and the placebo group. FIG. 10 is a graph of percentage of subjects experiencing pain relief versus time for the live device group and the placebo group. (This graph also provides separate data for “knee-pain only” subgroups of both the live device group and the placebo group.) FIG. 11 is a graph of percentage of subjects experiencing pain relief versus time for the placebo group and the follow-up study in which the placebo group was given live devices. FIG. 12 is a graph of duration of pain relief reported by subjects versus time for the live device group and the placebo group. FIG. 13 is a graph similar to FIG. 12 but also including the follow-up study in which the placebo group was given live devices.

This study demonstrated a trend of differences between live devices group and placebo group in terms of occurrence of relief and the duration of relief. The difference between baseline and 2 weeks follow up in the worst pain level (in the last 24 hrs) was highly statistically significant in the live devices group but not significant in the placebo group. The trend in the p-values suggests that with increase of the statistical power of the study (i.e. number of subjects) statistical significance may be achieved in the live devices group for other pain measures.

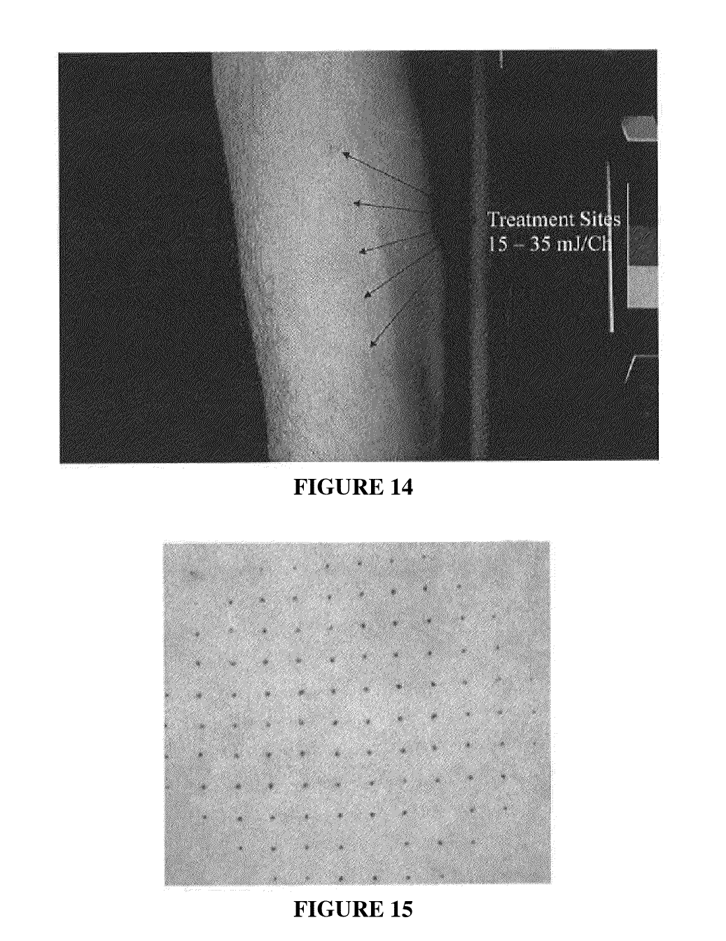

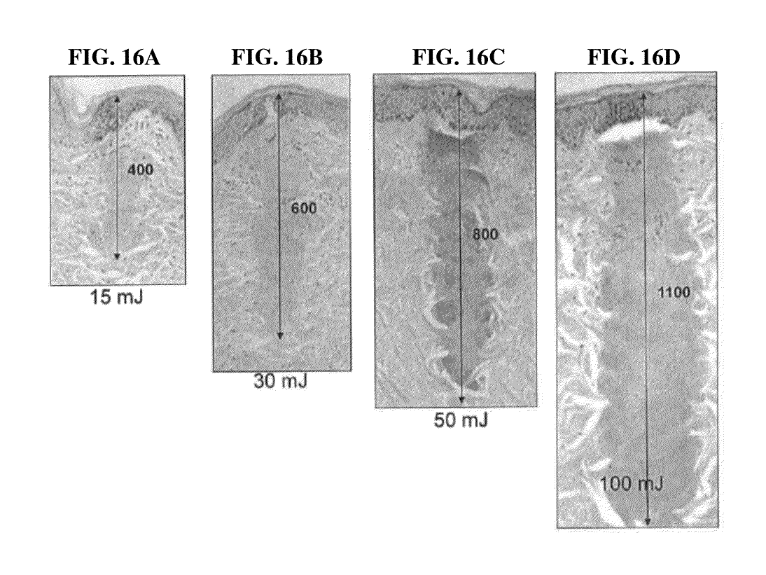

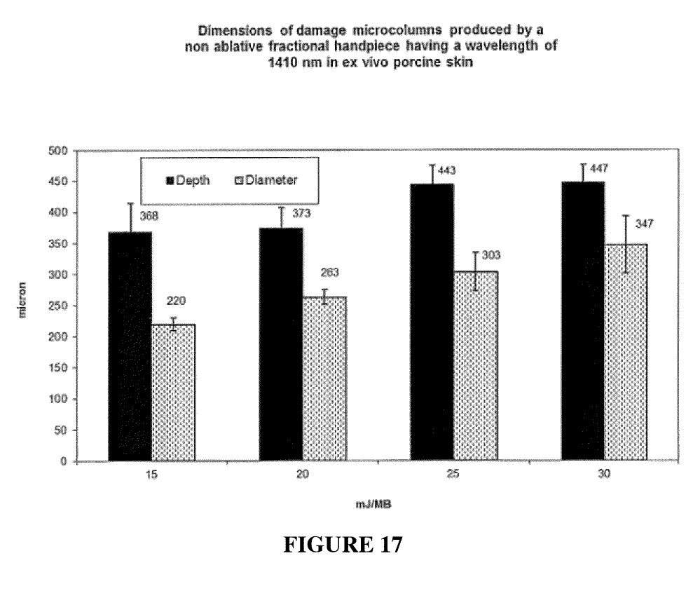

FIG. 14 is a photograph showing actual treatment sites on a subject's forearm. FIG. 15 is an enlargement photograph showing an array of micro islets formed at a treatment site. FIG. 16A is a cross-sectional microphotograph of a histological section slide showing the penetration depth of a single treatment column with 15 mJ applied to the treated islet at 1540 nm wavelength to achieve a penetration depth of 400 microns. FIG. 16B is a cross-sectional microphotograph of a histological section slide showing the penetration depth of a single treatment column with 30 mJ applied to the treated islet at 1540 nm wavelength to achieve a penetration depth of 600 microns. FIG. 16C is a cross-sectional microphotograph of a histological section slide showing the penetration depth of a single treat column with 50 mJ applied to the treated islet at 1540 nm wavelength to achieve a penetration depth of 800 microns. FIG. 16D is a cross-sectional microphotograph of a histological section slide showing the penetration depth of a single treat column with 100 mJ applied to the treated islet at 1540 nm wavelength to achieve a penetration depth of 1100 microns.

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

The patent, scientific and medical publications referred to herein establish knowledge that was available to those of ordinary skill in the art. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the claimed subject matter, the following definitions are provided for certain terms which are used in the specification and appended claims.

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

As used herein, unless specifically indicated otherwise, the word “or” is used in the inclusive sense of “and/or” and not the exclusive sense of “either/or.”

As used herein, EMR includes the range of wavelengths approximately between 200 nm and 20 mm. Optical radiation, i.e., EMR in the spectrum having wavelengths in the range between approximately 200 nm and 100 μm, is preferably employed in some of the embodiments described above, but, also as discussed above, many other wavelengths of energy can be used alone or in combination. Also as discussed, wavelengths in the higher ranges of approximately 2500-3100 nm may be preferable for creating micro-holes using ablative techniques. The term “narrow-band” refers to the electromagnetic radiation spectrum, having a single peak or multiple peaks with FWHM (full width at half maximum) of each peak typically not exceeding 10% of the central wavelength of the respective peak. The actual spectrum may also include broad-band components, either providing additional treatment benefits or having no effect on treatment. Additionally, the term optical (when used in a term other than term “optical radiation”) applies to the entire EMR spectrum. For example, as used herein, the term “optical path” is a path suitable for EMR radiation other than “optical radiation.”

It should be noted, however, that other energy may be used to for treatment islets in similar fashion. For example, sources such as ultrasound, photo-acoustic and other sources of energy may also be used to form treatment islets. Thus, although the embodiments described herein are described with regard to the use of EMR to form the islets, other forms of energy to form the islets are within the scope of the invention and the claims.

We claim:

1. A method for treating inflammation or pain, comprising:
determining a location of a volume of inflamed tissue; and
applying radiation suitable for damaging tissue to portions of the volume where a damaged portion of the volume is separated from another damaged portion by a non-treated portion of the volume.

2. The method of claim 1 further comprising introducing a corticotropin-releasing hormone to the body of the subject.

3. The method of claim 2 wherein the corticotropin-releasing hormone is in a topical.

4. The method of claim 3 wherein the topical is disposed on the location of the volume one of prior to applying radiation, after applying radiation, and simultaneous with applying radiation.

5. The method of claim 2 wherein the corticotropin-releasing hormone is injected into the location of the volume.

6. The method of claim 1 wherein the corticotropin-releasing hormone is ingested prior to applying radiation.

7. The method of claim 1 further comprising:
applying to portions of the volume radiation suitable for ablating tissue, where an ablated portion of the volume is separated from another ablated portion by an non-treated portion of the volume; and
disposing a corticotropin-releasing hormone to the location of the volume.

8. The method of claim 1 wherein the radiation is electromagnetic radiation.

9. The method of claim 1 wherein the radiation is one of optical radiation, ultrasound radiation, and radio frequency radiation.

10. The method of claim 1 wherein the volume of inflamed tissue is determined by the subject feeling that the location is tender to the touch.

11. A method for treating inflammation or pain, comprising:
applying radiation suitable for damaging tissue to portions of a volume of tissue, where a damaged portion of the volume is separated from another damaged portion by an non-treated portion of the volume; and
introducing a corticotropin-releasing hormone to the body of the subject.

12. The method of claim 11 wherein the corticotropin-releasing hormone is in a topical.

13. The method of claim 12 wherein the topical is disposed on the location of the volume one of prior to applying radiation, after applying radiation, and simultaneous with applying radiation.

14. The method of claim 12 wherein the corticotropin-releasing hormone is injected into the location of the volume.

15. The method of claim 12 wherein the corticotropin-releasing hormone is ingested prior to applying radiation.

16. The method of claim 11 wherein the radiation is electromagnetic radiation.

17. The method of claim 11 wherein the radiation is one of optical radiation, ultrasound radiation, and radio frequency radiation.

18. The method of claim 11 wherein the volume of inflamed tissue is determined by the subject feeling that the location is tender to the touch.

19. A method for treating chronic pain, comprising:
determining a location of a volume of inflamed tissue that is associated with the chronic pain; and
delivering non-ablative thermal tissue damage to a depth of at least 50 microns to at least a portion of the volume of inflamed tissue, to at least a portion of a volume of tissue adjacent the volume of inflamed tissue, or to at least a portion of a volume of tissue adjacent the volume of inflamed tissue and to at least a portion of the volume of inflamed tissue.

20. The method of claim 19 further comprising delivering the thermal tissue damage to a depth of at least 100 microns.

21. The method of claim 19 further comprising determining the need for additional pain treatment upon the resolution of the thermal tissue damage previously delivered.

22. The method of claim 19 wherein the thermal tissue damage portion is separated from another thermal tissue damage portion by a non-treated portion of the volume.

23. The method of claim 19 wherein the non-ablative thermal damage comprises cold treatment.

24. A device for treating inflammation or pain comprising:
an inflammation detector for detecting inflammation in a region of tissue;
a source of radiation configured to generate radiation to damage a volume of tissue; and
an optical path that delivers radiation from the source to the volume of tissue to form a damaged portion of the volume separated from another damaged portion of the volume by an non-treated portion of the volume.

25. The device of claim 24 wherein the inflammation detector comprises at least one of a thermometer, medical IR thermal camera, thermally sensitive film, video camera, and ultrasound inflammation detector.

26. The device of claim 24 wherein the optical path delivers radiation solely to the region of tissue containing inflammation.

27. The device of claim 24 wherein the inflammation detector signals the user to deliver radiation due to the presence of the region of tissue containing inflammation.

28. The device of claim 24 wherein the device is wearable and is removably attachable to a subject's body.

29. The device of claim 28 wherein the wearable device is one of a patch or a garment.

30. The device of claim 24 wherein at least one of the repetition rate of energy administration and the frequency of energy administration varies as a function of the detected inflammation.

31-33. (canceled)
